(19) United States
(12) Patent Application Publication
(10) Pub. No.: US 2016/0015289 A1
(43) Pub. Date: Jan. 21, 2016

(54) FORM FACTORS FOR THE MULTI-MODAL PHYSIOLOGICAL ASSESSMENT OF BRAIN HEALTH

(71) Applicants: Adam J. SIMON, Yardley, PA (US); Gary S. KATH, Scotch Plains, NJ (US); Cerora, Inc., Bethlehem, PA (US)

(72) Inventors: Adam J. SIMON, Yardley, PA (US); Gary S. KATH, Scotch Plains, NJ (US)

(21) Appl. No.: 14/772,996

(22) PCT Filed: Mar. 6, 2014
(86) PCT No.: PCT/US14/21247

§ 371 Date: Sep. 4, 2015

Related U.S. Application Data
(60) Provisional application No. 61/773,428, filed on Mar. 6, 2013.

(51) Int. Cl. A61B 5/0484; A61B 5/0478; A61B 5/16; A61B 7/04; A61B 5/026; A61B 3/14; A61B 5/00; A61B 5/04; A61B 5/1455; A61B 5/0205; A61B 3/113

(52) U.S. Cl. CPC A61B 5/04842; A61B 5/02055; A61B 5/0478; A61B 5/162; A61B 7/04; A61B 3/113; A61B 3/145; A61B 5/04845; A61B 5/04847; A61B 5/0057; A61B 5/0006; A61B 5/04012; A61B 5/7203; A61B 5/7246; A61B 5/7264; A61B 5/6898; A61B 5/14552; A61B 5/026; A61B 5/4041; A61B 5/6803; A61B 2562/0219; A61B 2560/0475; A61B 2560/0443

(57) ABSTRACT

A multi-modal physiological assessment device and method enables the simultaneous recording and then subsequent analysis of multiple data streams of biological signal measurements to assess the health and function of the brain. The multi-modal assessment system includes at least one channel of EEG brainwave data in combination with cognitive information that provide a two-dimensional data stream of (x(t), y(t)) of cognitive information; voice recordings; motion, position, and stability data; galvanic skin conductance; temperature of the subject; pulse-oximetry data, cerebral blood perfusion data, vaso-motor reactivity data, and the like. The collected data is processed to construct candidate features extracted from multiple biological sensor data streams and correlated with multi-modal signatures to identify data indicative of brain health, disease and injury.

### CROSS REFERENCE TO RELATED APPLICATIONS

This application claims benefit of Provisional Application No. 61/773,428 filed Mar. 6, 2013. The content of that patent application is hereby incorporated by reference in its entirety.

### TECHNICAL FIELD

The invention relates to diagnosis and analysis of brain health through the use of activated tasks and stimuli in a system to dynamically assess one's brain state and function.

### BACKGROUND

Normal functioning of the brain and central nervous system is critical to a healthy, enjoyable and productive life. Disorders of the brain and central nervous system are among the most dreaded of diseases. Many neurological disorders such as stroke, Alzheimer's disease, and Parkinson's disease are insidious and progressive, becoming more common with increasing age. Others such as schizophrenia, depression, multiple sclerosis and epilepsy arise at younger age and can persist and progress throughout an individual's lifetime. Sudden catastrophic damage to the nervous system, such as brain trauma, infections and intoxications can also affect any individual of any age at any time.

Most nervous system dysfunction arises from complex interactions between an individual's genotype, environment and personal habits and thus often presents in highly personalized ways. However, despite the emerging importance of preventative health care, convenient means for objectively assessing the health of one's own nervous system have not been widely available. Therefore, new ways to monitor the health status of the brain and nervous system are needed for normal health surveillance, early diagnosis of dysfunction, tracking of disease progression and the discovery and optimization of treatments and new therapies.

Unlike cardiovascular and metabolic disorders, where personalized health monitoring biomarkers such as blood pressure, cholesterol, and blood glucose have long become household terms, no such convenient biomarkers of brain and nervous system health exist. Quantitative neurophysiological assessment approaches such as positron emission tomography (PET), functional magnetic resonance imaging (fMRI) and neuropsychiatric or cognition testing involve significant operator expertise, inpatient or clinic-based testing and significant time and expense. One potential technique that may be adapted to serve a broader role as a facile biomarker of nervous system function is a multi-modal assessment of the brain from a number of different forms of data, including electroencephalography (EEG), which measures the brain's ability to generate and transmit electrical signals. However, formal lab-based EEG approaches typically require significant operator training, cumbersome equipment, and are used primarily to test for epilepsy.

Alternate and innovative biomarker approaches are needed to provide quantitative measurements of personal brain health that could greatly improve the prevention, diagnosis and treatment of neurological and psychiatric disorders. Unique multi-modal devices and tests that lead to biomarkers of Parkinson's disease, Alzheimer's disease, concussion and other neurological and neuropsychiatric conditions is a pressing need.

### SUMMARY

The invention provides a system and method to address the above needs in the art by capturing multiple streams of biological sensor data for assessing brain health of a user. In an exemplary embodiment, the system includes a plurality of biological sensors adapted to collect biological sensor data from the user. The biological sensors include an active brainwave sensor that collects at least one channel of EEG brainwave data, and at least one of the following:

an accelerometer and/or a gyrometer that collects motion, position, and stability data to provide quantitative stability and balance measurements,

a peripheral sensing device that collects cognitive information in the form of neuropsychological data comprising key board keystrokes, mouse clicks, and/or touch panel events to convey reaction time and accuracy information,

a microphone that records human speech to capture verbal responses of the human subject during a battery of tasks to either cognitive challenges or auditory stimulations,

a camera or biosensor that records that records eye movements, eye saccade and other biometric identification information;

a heart rate sensor that monitors heart rate,

a pulse oximeter that measures arterial oxygenation,

a temperature sensor that measures body temperature, and

a galvanic skin response or electrodermal response sensor that measures skin surface galvanic skin conductance and/or electrical skin resistance.

An electronic module that is, for example, incorporated into a disposable headband, simultaneously records the biological sensor data collected by the plurality of biological sensors and transmits the collected biological sensor data to a server for processing. A stimulation device further applies at least one of a visual stimulant, an auditory stimulant, a gastronomic stimulant, an olfactory stimulant, and/or a motion stimulant to the user. During operation, the plurality of biological sensors simultaneously measure the body's response to stimulants applied by the stimulation device for recordation by the electronic module.

In exemplary embodiments, the server processes biological sensor data received from the electronic module to identify and characterize artifacts, to extract candidate features for classification and storage and/or for comparison to previously acquired candidate features, and to generate a report. The server also may build extracted biometric tables from candidate features extracted from the received biological sensor data. The server also may be programmed to construct predictive signatures including candidate features extracted from multiple biological sensor data streams. In exemplary embodiments, the predictive signatures correlate EEG data and cognitive and/or data from any of the other data streams with multi-modal signatures of brain health, disease and injury.

In further exemplary embodiments, the peripheral sensing device, microphone, and camera or biosensor are implemented in a PC, tablet PC, smartphone or custom hand held device that is programmed to administer instructions to the user via a sound card and/or visual display of the PC, tablet PC, smartphone or custom hand held device. The PC, tablet PC, smartphone or custom hand held device also may be programmed to provide control signals to the stimulation device.

Another aspect of the invention is the use of a simple disposable head band and electrodes to enable use of an electronics module multiple times without human contact and possible contamination. Embodiments of the invention include fiber optics or light pipes in an ear clip or surface patch to enable simultaneous EEG and Pulse-Oximetry. In another embodiment, simultaneous temperature is included along with EEG. In yet another embodiment, accelerometers are used not to measure the position of the head but rather as another biological signal to signify motion and stability during balance and vestibular tasks while EEG is being recorded, thus enabling the extraction of features from each data stream including the possibility to create a cross-correlation between any two time synchronized data streams.

In one embodiment of the invention, the use of a reusable electronic module (REM) and an electrode without the use of a wire, but directly snapped or mechanically and electrically connected, makes for a compact and efficient REM module. Also a part of this embodiment are single and dual channel adhesive electrodes or an insert into an ear clip to serve as a disposable item.

An additional embodiment of the invention uses a built in vibrational oscillator to calibrate a measurement accelerometer remotely before each use to ensure system and sensor reliability, much like test signals are applied to an electronic circuit.

An additional embodiment of the invention includes various means to take temperature either from the forehead of the human subject or through the mouth in a fashion that is connected to the body worn electronic module. In one embodiment, a temperature sensor is placed in the ear canal while the EEG ear clip is held in place from one and the same mechanical unit.

Additional embodiments of the invention include the ability to provide gastronomic and olfactory stimulation in an automated fashion, programmed from the body worn REM while recording the data streams of biological signals in a parallel and time synced fashion.

Additional embodiments include the construction of predictive signatures that include features extracted from multiple biological signal data streams to make signatures with increased sensitivity and specificity, including use of cognitive measures like KD Total time and EEG relative beta power.

The invention also includes methods for measuring biological data using such devices. These and other characteristic features of the invention will become apparent to those skilled in the art from the following description of the exemplary embodiments.

### BRIEF DESCRIPTION OF THE DRAWINGS

Embodiments of the invention can be better understood with reference to the following drawings.

FIG. 1 is a schematic diagram illustrating the simplified headband based REM system to record a single channel of EEG.

FIG. 2 is a top down view schematic diagram illustrating the mounting of the REM to the headband showing the active electrode snapped into place on the inside of the headband.

FIG. 3A is a schematic diagram illustrating a transmission based pulse oximetry ear clip that enables both EEG and pulse oximetry from the same REM and ear clip.

FIG. 3B is a schematic diagram illustrating a reflection based pulse oximetry ear clip that enables both EEG and pulse oximetry from the same REM and ear clip.

FIG. 4 is a schematic illustration of a disposable ear clip insert.

FIG. 5 is a schematic illustration of a disposable ear clip insert just before being inserted into an ear clip.

FIG. 6 is a schematic illustration of a disposable ear clip just after it has been inserted into an ear clip, ready for use on a human subject's ear.

FIG. 7 is a schematic illustration of a two channel adhesive electrode for reference REF and ground GND.

FIG. 8 is a schematic illustration of a headband with alternate electrode placement.

FIG. 9 is a schematic illustration of an exploded view of the headband of FIG. 8 with alternate electrode placement.

FIG. 10 is a schematic illustration of a headband supported electronics module with adhesive ear electrodes on the frame of a model head.

FIG. 11 is a schematic illustration of a headband supported electronics module with adhesive ear electrodes allowing view of the active electrode on the inside of the headband immediately behind the electronics module.

FIG. 12 is a 3D front view drawing of an electronics module.

FIG. 13 is a 3D rear view drawing of an electronics module.

FIG. 14 is a 3D exploded front view drawing of an electronics module.

FIG. 15 is a 3D exploded rear view drawing of an electronics module.

FIG. 16 is a 3D compact rear view drawing of an electronics module.

FIG. 17A and FIG. 17B together schematically illustrate a headband supported electronics module with an adjacent temperature sensor for concurrent EEG and temperature based measurements.

FIG. 18 is a schematic illustration of an REM module including a mouth inserted temperature probe protected with a disposable sheath.

FIG. 19A is a schematic diagram of a thermistor temperature sensor interfaced to an REM module.

FIG. 19B is a schematic diagram of an analog temperature sensor interfaced to an REM module.

FIG. 20 is a schematic diagram of a digital temperature sensor interfaced to an REM module.

FIG. 21A is a schematic diagram of an ear canal temperature sensor using a spot IR temperature sensor for interfacing to an REM module.

FIG. 21B is a schematic diagram of a spot IR temperature sensor interfaced to an REM module.

FIG. 22 is a schematic diagram of a multi-point imaging IR sensor interfaced to an REM module.

FIG. 23 is a schematic illustration of a peripheral finger mounted REM module.

FIG. 24 is a schematic illustration of a peripheral wrist or ankle mounted REM module.

FIG. 25 is a schematic illustration of a gastronomic stimulant apparatus controlled by an REM module and delivering stimulus to the mouth a subject.

FIG. 26 is a schematic illustration of a single-fluid gated solenoid gastronomic stimulant apparatus connected to an REM module.

FIG. 27 is a schematic illustration of a multi-fluid gated solenoid gastronomic stimulant apparatus connected to an REM module.

FIG. 28 is a schematic illustration of an olfactory stimulant apparatus inserted into a patient's nose and controlled via an REM module.

FIG. 29 is a schematic illustration of a “Scratch & Sniff” olfactory stimulant apparatus connected to an REM module.

FIG. 30 is a schematic illustration of a multi-modal brain health assessment system including 1) an REM module that collects single lead EEG brainwave data that is transmitted to a tablet via Bluetooth; 2) a peripheral mobile computing unit (MCU) including touch screen “events” that convey cognitive data (in the form of reaction time Rx and accuracy); 3) voice data recorded via the built-in tablet microphone; 4) image data from the front facing built-in camera or biosensor enabling biometric identification and other image processing analysis including eye movement tracking such as saccade; 5) built-in accelerometer, gyrometer and magnetic compass that enables assessment of balance and stability; and 6) other built-in sensors that provide other data streams to the system.

FIG. 31 is a schematic illustration of enterprise cloud based activities for processing the collected data streams, which include signal pre-processing, signal processing, biometric feature table construction, predictive analytics, and report generation.

FIG. 32 is a schematic illustration of the full lifecycle of a diagnostics as a service product/service mix.

FIG. 33 is a graphical display of the calibration measurements from a 3-axis accelerometer hung on the end of string and oscillated as a suspension pendulum.

FIG. 34 is a graphical display of a 3-axis accelerometer affixed to the wrist on a human subject walking while swinging their arm as they navigate an obstacle course within an office environment.

FIG. 35 is a pair of graphical displays of a logistic plot and its corresponding Receiver Operating Characteristic curve (ROC) of an EEG feature (relative beta) used to predict the clinical diagnosis of concussion subjects versus control subjects.

FIG. 36 is a pair of graphical displays of the Receiver Operating Characteristic curve (ROC) of an EEG feature (relative beta) combined with a cognitive task score from the King-Devick test as a pair or in combination with two co-variates, age and gender.

FIG. 37A is schematic illustration of an alternate form factor for a headband to support or hold an REM on the head in the form of a glasses frame without the lenses.

FIG. 37B is schematic illustration of an alternate form factor which consists of disposable ear temple supports and disposable nose pads, both of which touch the human and support or hold an REM on the head in the form of a glasses frame without the lenses.

FIG. 37C is schematic illustration of an alternate form factor for a headband to support or hold an REM on the head in the form of a glasses frame without the lenses with integrated wires, a channel or key to slide the REM along, and the means to connect skull and mastoid electrodes.

FIG. 38 is schematic illustration of an alternate REM which is either supported on the arm of an subject or around their waist which has long leads that allow more support during rest or sleep based data gathering.

### DETAILED DESCRIPTION OF ILLUSTRATIVE EMBODIMENTS

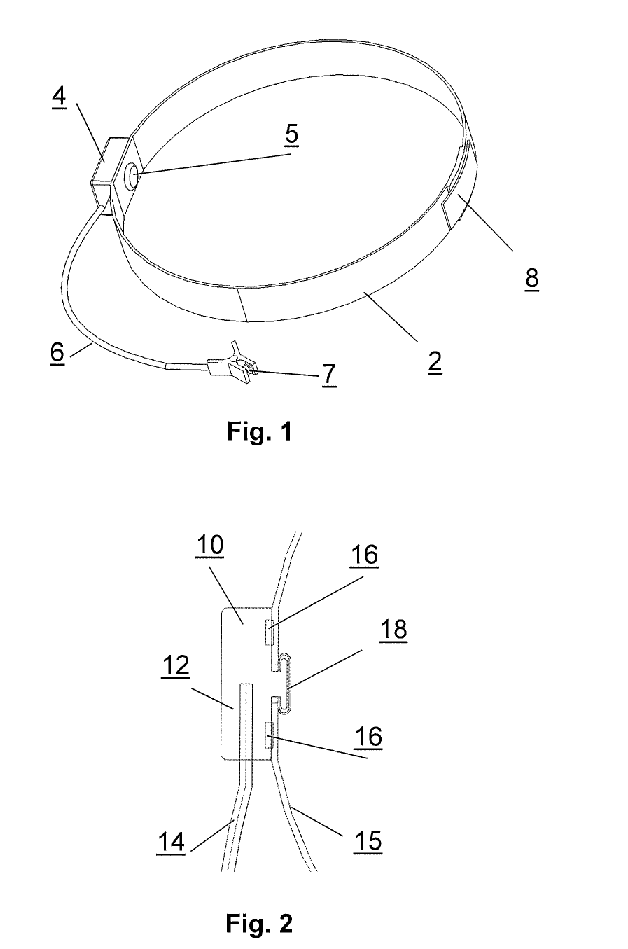

The invention will be described in detail below with reference to FIGS. 1-38. Those skilled in the art will appreciate that the description given herein with respect to those figures is for exemplary purposes only and is not intended in any way to limit the scope of the invention. All questions regarding the scope of the invention may be resolved by referring to the appended claims.

### DEFINITIONS

By “electrode to the scalp” we mean to include, without limitation, those electrodes requiring gel, dry electrode sensors, contactless sensors and any other means of measuring the electrical potential or apparent electrical induced potential by electromagnetic means.

By “monitor the brain and nervous system” we mean to include, without limitation, surveillance of normal health and aging, the early detection and monitoring of brain dysfunction, monitoring of brain injury and recovery, monitoring disease onset, progression and response to therapy, for the discovery and optimization of treatment and drug therapies, including without limitation, monitoring investigational compounds and registered pharmaceutical agents, as well as the monitoring of illegal substances and their presence or influence on an individual while driving, playing sports, or engaged in other regulated behaviors.

A “medical therapy” as used herein is intended to encompass any form of therapy with potential medical effect, including, without limitation, any pharmaceutical agent or treatment, compounds, biologics, medical device therapy, exercise, biofeedback or combinations thereof.

By “EEG data” we mean to include without limitation the raw time series, any spectral properties determined after Fourier transformation, any nonlinear properties after non-linear analysis, any wavelet properties, any summary biometric variables and any combinations thereof.

A “sensory and cognitive challenge” as used herein is intended to encompass any form of sensory stimuli (to the five senses), cognitive challenges (to the mind), and other challenges (such as a respiratory CO₂ challenge, virtual reality balance challenge, hammer to knee reflex challenge, etc.).

A “sensory and cognitive challenge state” as used herein is intended to encompass any state of the brain and nervous system during the exposure to the sensory and cognitive challenge.

An “electronic system” as used herein is intended to encompass, without limitation, hardware, software, firmware, analog circuits, DC-coupled or AC-coupled circuits, digital circuits, FPGA, ASICS, visual displays, audio transducers, temperature transducers, olfactory and odor generators, or any combination of the above.

By “spectral bands” we mean without limitation the generally accepted definitions in the standard literature conventions such that the bands of the PSD are often separated into the Delta band (f<4 Hz), the Theta band (4<f<7 Hz), the Alpha band (8<f<12 Hz), the Beta band (12<f<30 Hz), and the Gamma band (30<f<100 Hz). The exact boundaries of these bands are subject to some interpretation and are not considered hard and fast to all practitioners in the field.

By “calibrating” we mean the process of putting known inputs into the system and adjusting internal gain, offset or other adjustable parameters in order to bring the system to a quantitative state of reproducibility.

By “conducting quality control” we mean conducting assessments of the system with known input signals and verifying that the output of the system is as expected. Moreover, verifying the output to known input reference signals constitutes a form of quality control which assures that the system was in good working order either before or just after a block of data was collected on a human subject.

By “biomarker” we mean an objective measure of a biological or physiological function or process.

By “biomarker features or metrics” we mean a variable, biomarker, metric or feature which characterizes some aspect of the raw underlying time series data. These terms are equivalent for a biomarker as an objective measure and can be used interchangeably.

By “non-invasively” we mean lacking the need to penetrate the skin or tissue of a human subject.

By “diagnosis” we mean any one of the multiple intended use of a diagnostic including to classify subjects in categorical groups, to aid in the diagnosis when used with other additional information, to screen at a high level where no a priori reason exists, to be used as a prognostic marker, to be used as a disease or injury progression marker, to be used as a treatment response marker or even as a treatment monitoring endpoint.

By “electronics module” or “EM” or “reusable electronic module” or “REM” or “multi-functional biosensor” or “MFB” we mean an electronics module or device that can be used to record biological signals from the same subject or multiple subjects at different times. By the same terms, we also mean a disposable electronics module that can be used once and thrown away which may be part of the future as miniaturization becomes more common place and costs of production are reduced. The electronics module can have only one sensing function or a multitude (more than one), where the latter (more than one) is more common. All of these terms are equivalent and do not limit the scope of the invention.

### Simplified Form Factor for the Acquisition of a Multiple Streams of Biological Signal Data in the Assessment of Brain Health and Function

The systems and methods of the invention comprise device and equipment form factors that can easily be positioned on the human body to both stimulate various senses as well as collect a multitude of bio-signals, can be re-used in part and disposed in part, and utilized locally using personalized and disposable materials when they touch the human body. It is often necessary to insure the integrity and sterility of any item that comes in contact with a human test subject by either disinfecting the applied part or dispensing of the previous one and using a fresh and unused set of materials that come in contact with the human subject. Moreover, it would be advantageous to have a minimal cost associated with the disposable parts that get thrown out as waste into a trash can.

A solution to these problems includes the creation of one or more electronic modules or multi-functional biosensors (MFB) that can be placed on the body to record bio-signals from the body. In particular, one such electronic module (EM) can be placed in the vicinity of the head and be either reused over and over if it does not touch the human body or disposed of if it comes in direct contact with the human body.

In one embodiment as illustrated in FIG. 1, a form factor of the invention includes a headband 2, which supports an electronic module or reusable electronic module (REM) 4, which has an active brainwave sensor 5 that sits directly on the forehead. The differential input signal is contacted to a non-skull portion of the body, preferably someplace easy to access like the earlobe or top of the ear off of the skull through cable 6 to ear clip 7 which includes either one conductor or two conductors, one for Reference (REF) and the other for Ground (GND). The REM 4 and the active brainwave sensor 5 can be attached through a common medical device electronic snap or other simple press electro-mechanical connection. The REM 4 and cable 6 can be attached to the headband 2 via Velcro hook/ladder press closure as well. Alternate designs of ear clip 7 are a part of the invention and they will be described later in further detail. At the back of the headband, a piece of Velcro or similar press fit closure 8 can be used to secure the headband to the human subject's head with a secure but comfortable tight mechanical fit. In an exemplary embodiment, the head band 2 is made from Fabrifoam's unique fabric-foam dual layer material which stretch easily and is very comfortable to sit on the skin because of the water permeation properties of the material.

In FIG. 2, a top down view is shown of the REM 10 attached to headband 15 by Velcro tabs 16 to head band 15. In addition, electrode 18 is attached to REM 10 via a button snap mechanical closure which goes thru a hole punched in headband 15 for this purpose. Electrode 18 can be made of silver, gold, stainless steel, or various dry gel or wet gel silver/silver chloride electrode sensors available from firms such as 3M (Reddot) or Vermed (NeuroPlus). This hole provides a means to both secure the REM 10 to the head band 15 and also to enable a direct electrical connection from the human subject's forehead to the active input of the EEG analog front end in the REM 10. Remote cable 12 connects the internal electronics of the REM 10 inside via external cable 14 to the ear or other mastoid location. The remote mastoid cable 12 inside and 14 outside the REM 10 can be electronic in nature only or, in other embodiments of the invention, may include optical fibers to carry and return light for contemporaneous pulse-oximetry based measurements.

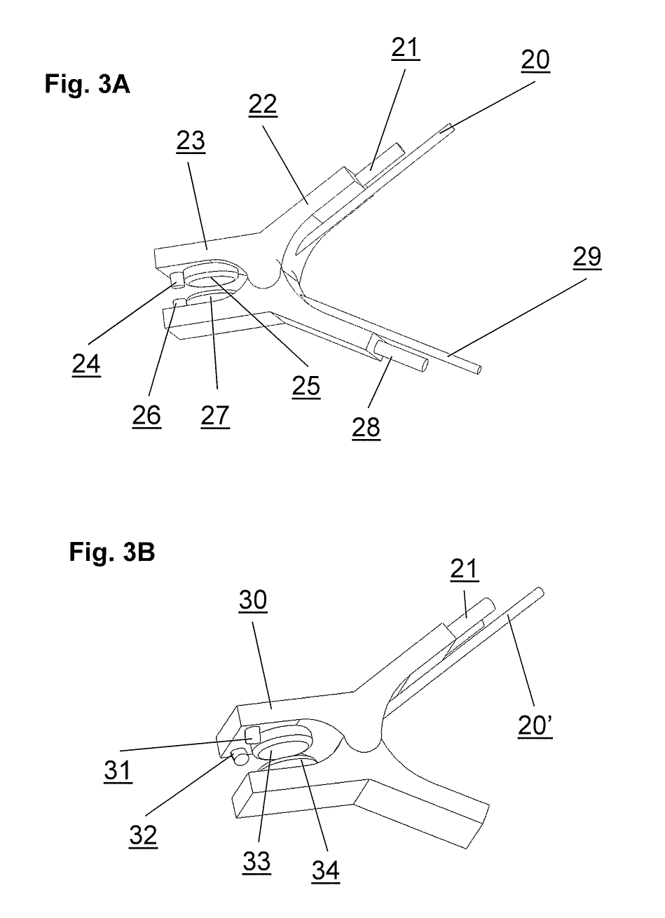

FIG. 3A illustrates an embodiment of the invention where the ear clip has been transformed to include not only electrical contacts for REF and GND, but also includes means to simultaneously measure heart rate and arterial oxygen (Pulse-Oximetry). In FIG. 3A, electrical connections of Reference REF 21 and ground GND 28 are similar to before, but now there are two fiber optic cables 20 and 29 delivering light from an LED in the REM 10 on the forehead to the ear clip via light pipe 20 which presents the light in transmission mode to the ear through the plastic clip 22 to the upper armature 23 which holds REF electrical contact 25 and light source output 24. On the contralateral ear clip armature (designed for the ear lobe), light pipe input 26 collects the transmitted light through the ear and returns it through fiber optic 29 to a photodiode in the REM 10. Electrical contact 27 makes contact as Ground GND. In this way, the simple attachment of the ear clip provides simultaneous dual lead electrical contact as well as input/output for the LED light source and photodiode light detector for pulse-oximetry based measurements.

FIG. 3B illustrates an alternate embodiment for reflection mode pulse-oximetry rather than transmission mode pulse oximetry. Here, electrical contact for REF and GND are made through dual conductor cable 20′ to electrical contacts 33 and 34, similar to FIG. 3A. However, in this case, dual light pipes are situated on the same side of ear clip armature 30 so that illumination is via light pipe output 31 where the reflected light is measured by light pipe input 32. In either example, pulse-oximetry for heart rate and arterial oxygen is conducted simultaneously with the use of the electrical mastoid ear clip REF and GND. A similar embodiment could be done directly on the REM with holes or windows in the headband without the use of the fibers for light transmission and detection.

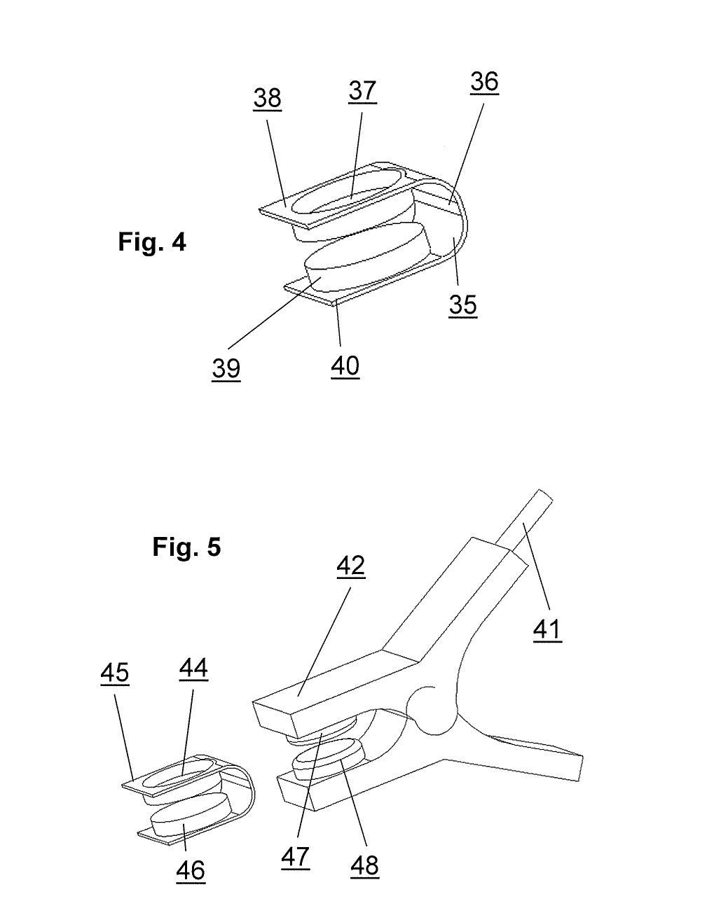

In order to provide for a device in which any part that touches the human can be made disposable, a disposable insert is provided as illustrated in FIG. 4. Substrate 35 is folded in half on itself where electrical contact 37 can be made to REF while electrical contact 39 can be made to GND. The substrate is either made of an insulator or there is an insulating barrier 36 between the top half 38 and the bottom half 40 of the disposable insert.

In FIG. 5, one sees the ear clip disposable insert 45 with disposable REF electrode 44 and disposable GND electrode 46 about to get inserted into ear clip 42 connected to dual lead cable 41 with fixed REF electrode 47 and fixed GND electrode 48.

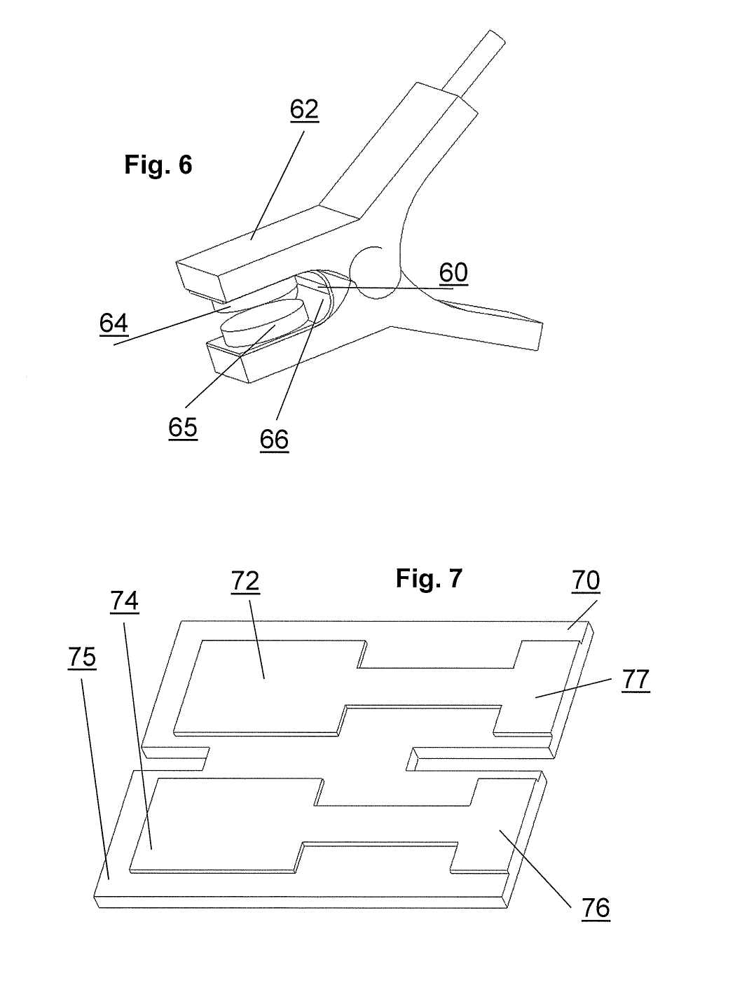

FIG. 6 illustrates a disposable ear clip insert 66 with disposable REF electrical contact 64 and disposable GND electrical contact 65 that has been placed into ear clip 62, which includes electrical insulator 60 designed to isolate each of REF 64 and GND 65.

In an alternate embodiment of the invention, instead of using a spring loaded ear clip to create a mechanical connection that provides a dual electrical REF and GND electrical connection to a mastoid, an adhesive mechanical approach is possible to the same end. As shown in FIG. 7, two isolated electrical conductors 72 for REF and 74 for GND can be deposited onto insulating substrates 70 and 75, which can be one and the same or two separate substrates then mechanically held together. The electrodes 72 and 74 can be coated by well-known dry gel or wet gels to make electrical contact with the skin. Two single or one dual lead alligator style clip can be attached to the dual channel adhesive electrode at tabs 76 for GND and 77 for REF. One skilled in the art can imagine alternate adhesive electrode configurations as well.

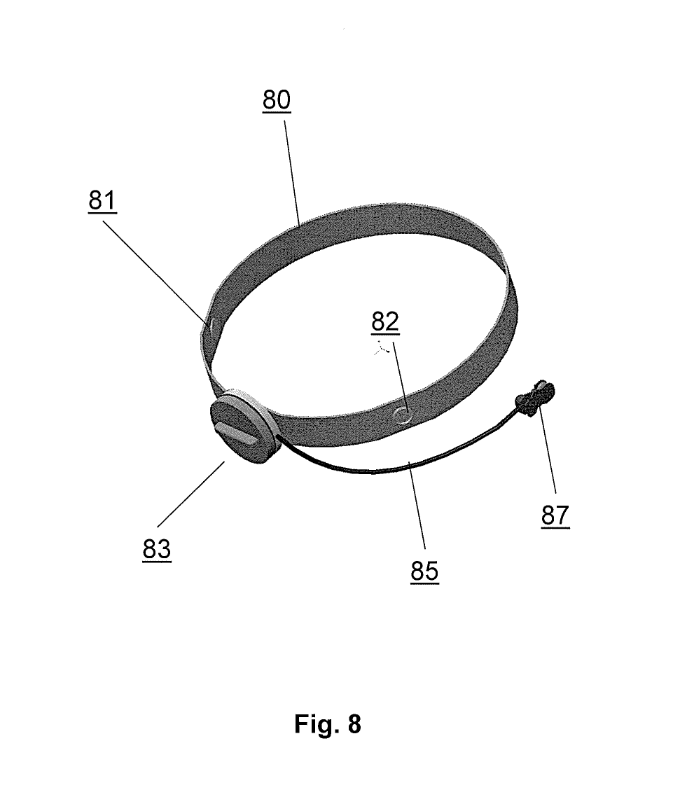

In another alternate embodiment, shown in FIG. 8, headband 80 has REM 83 attached as before but now there are additional electrodes such as on the temple 81 and or otherwise located around the head 82 and attached to the headband 80. In this embodiment, two, three or four channels of EEG data can be recorded to monitor both hemispheres of the brain as well as other spatial locations. Interconnect cable 85 and ear clip 87 for REF and GND ear contact are as described before.

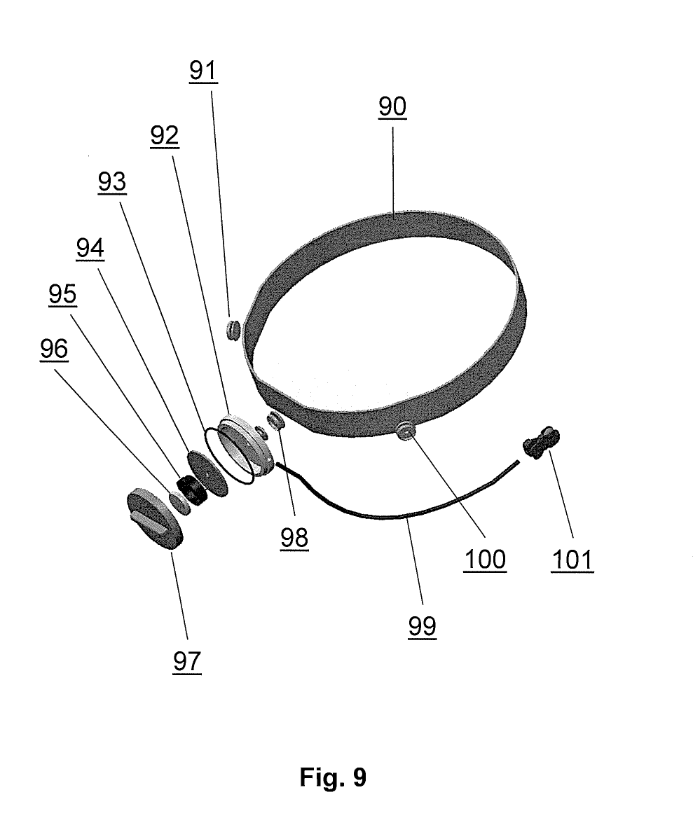

FIG. 9 provides an exploded view of the REM 83 with headband 90 holding temple snap electrode 91, active forehead snap electrode 98 and alternate location snap electrode 100. Enclosure 92 is held with 0-ring 93, printed circuit board 94, and battery holder 95, which holds coin cell battery 96. The entire package is contained via cap 97 which mates with enclosure 92. Ear interconnect cable 99 enables ear clip 101 to make electrical contact for REF and GND to the ear or mastoid.

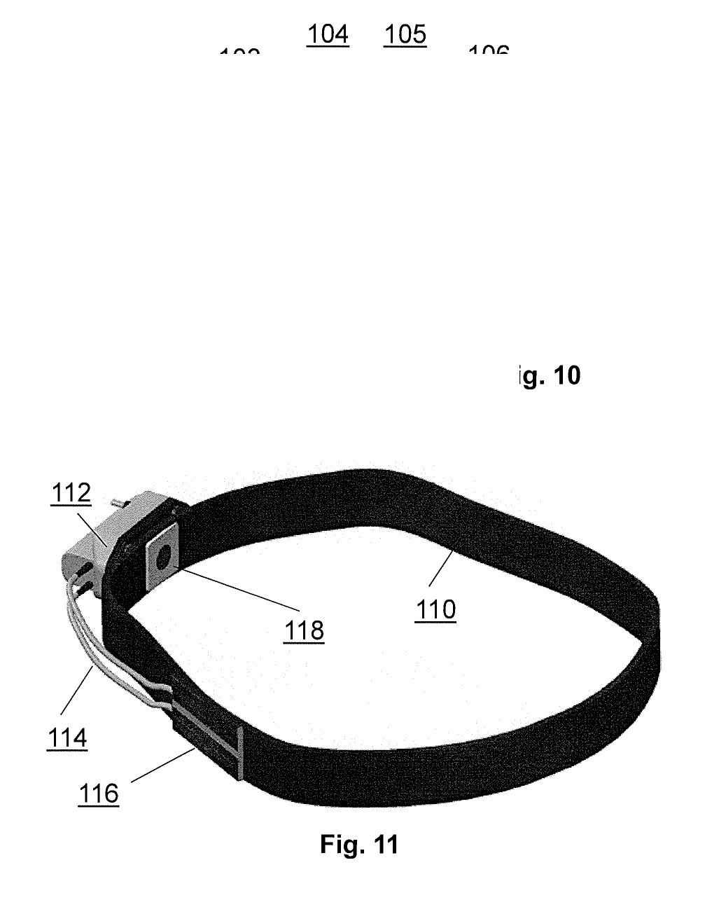

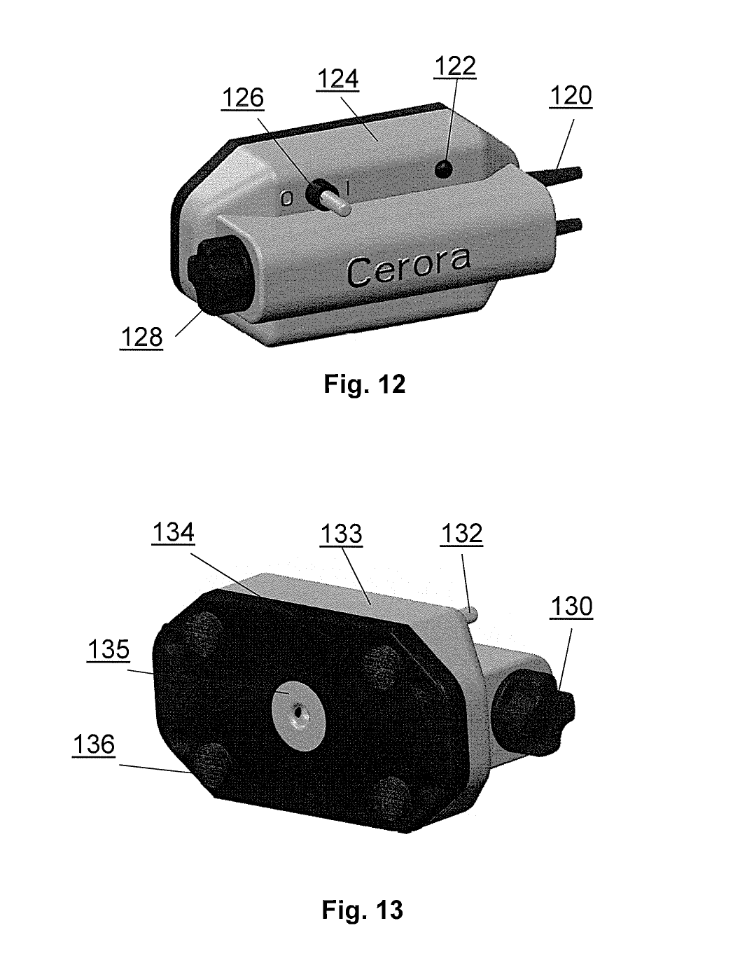

In the embodiment of the invention show in FIG. 10, the REM 104 is fixed via Velcro-like hook/ladder press closures to headband 103. Attached to the upper ear inner surface is dual contact adhesive electrode 106 attached electrically by one dual lead cable or two single lead cables 105 back to the internal electronics of REM 104. In this example, a size AAA battery holder is visible to enable sufficient power for long term wireless monitoring of the subject in the case of ambulatory monitoring applications. Where power is reduced and longevity is not needed, coin cell batteries can be used. As shown in FIG. 11 off of the human subject's head, the disposable headband 110 holds disposable active electrode 118 on the inside snug against the human subject's forehead for a strong secure mechanical and electrical connection. REM 112 connects via ear interconnect cable 114 to disposable ear clip 116. Viewed in isolation as a full assembly from the front, FIG. 12 shows the REM 124 to have an on/off/pair switch 126, a removable battery loading screw cap 128, an indicator LED 122 and ear interconnect cable 120.

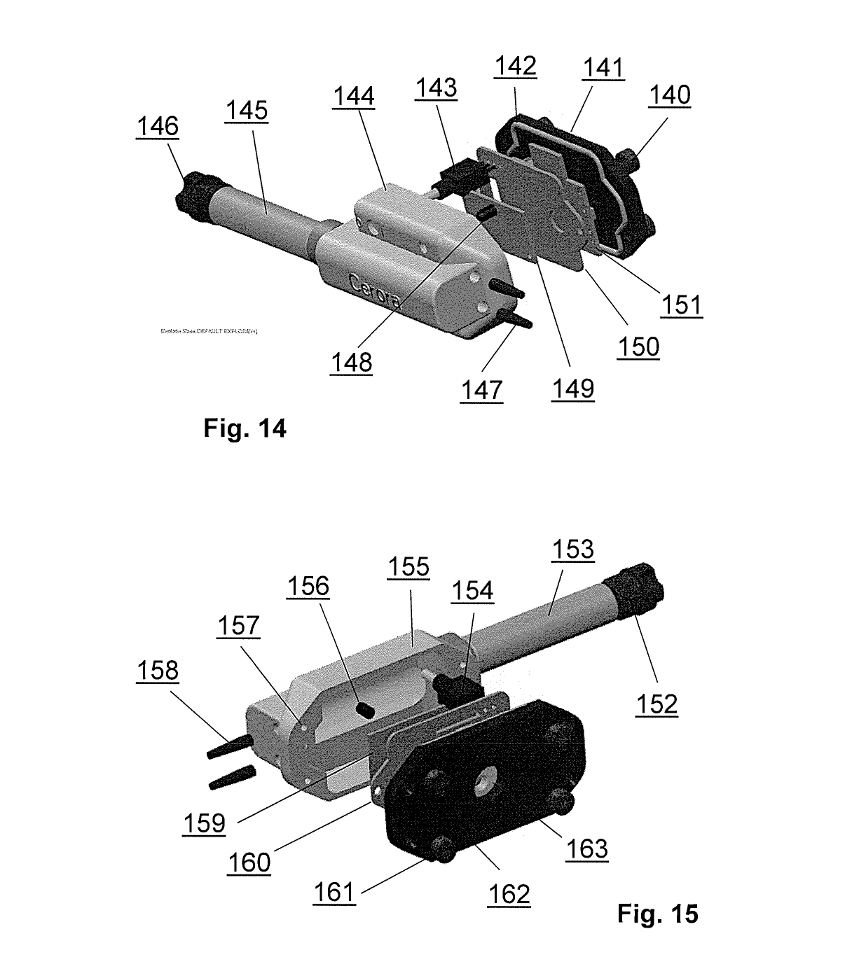

From the rear, shown in FIG. 13, the REM 133 shows removable battery compartment cap 130, switch 132, rear plate 134, Velcro press closure pads 136, and a snap 135 to both mechanically and electrically make contact with the forehead active electrode. As shown in FIG. 14, in a front side exploded view, the battery enclosure cap 146 sits next to AAA battery 145 in REM front housing 144. Switch 143 attaches to PCB 150 with LED 148 mounted on PCB 149 which sandwich together with PCB 151. Interconnect cables 147 connect to the ear clip for reference REF and Ground GND. O-ring 142 makes a water tight IP-67 seal between rear housing back plate 141 and front enclosure 144. Velcro closure pads 140 enable firm grip to the head band not shown in addition to the mechanical stability provided by the snap electrode to active forehead electrode.

In an alternate view shown in FIG. 15 from the rear, rear enclosure plate 162 holds Velcro closure pads 161 and allows direct snap 163 connections to the active forehead electrode. PCBs 159 and 160 hold switch 154 and LED 156 while battery 153 is retained by battery enclosure cap 152. Ear interconnect cable 158 connects the electronics within REM 155 to the mastoid. Screw hole 157 enables mechanical assembly under compression of the entire unit.

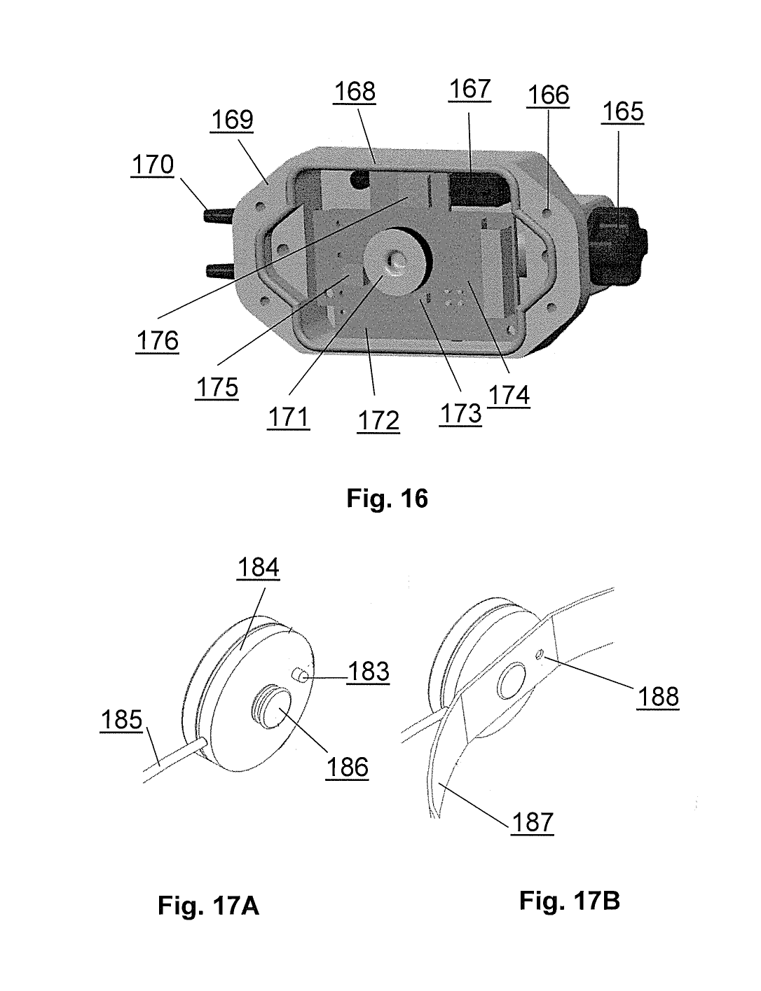

In the last view shown in FIG. 16, battery enclosure cap 165 sits in REM 169 with O-ring 168 making a water tight seal. Switch 167, screw hole 166, and ear interconnect cable 170 can be seen. Rear electrode snap 171 connector enables mechanical and electrical connection to the active forehead electrode. Bio sensor PCB module 172 can be seen with various biological sensor detectors and integrated circuits (ICs) such as EEG sensor 175, temperature sensor 173, 3-axis accelerometer 176, and pulse-oximeter IC 174.

### Additional Sensors and Biological Signals to be Measured and Monitored Beyond EEG Brainwaves

In addition to the EEG signals being measured and recorded, either locally within the REM, or transmitted via a wireless link for data capture and analysis, additional medical sensors (i.e. temperature, heart rate, etc.) may be connected to the EEG headband reusable electronics module (REM) to enhance the subject's evaluation and assessment. Such sensors are described below.

### Temperature Sensors

Thermistor Temperature Sensing

As known to those skilled in the art, a thermistor is a type of resistor whose resistance varies with temperature. Various size positive temperature coefficient (PTC) and negative temperature coefficient (NTC) thermistors are commercially available with a variety of resistance vs. temperature profiles. Miniature thermistors provide fast thermal response times of less than 1 second.

FIG. 17A shows one method of using a thermistor sensor to measure the human subject's forehead temperature and record the temperature signal via the headset's REM. The thermistor 183 is mounted to the exterior of the headband's REM enclosure 184 adjacent to the EEG forehead active sensor 186 in opposition to REF and GND from ear interconnect cable 185. The protruding portion of the thermistor would mate to a hole cut-out on the elastic headband 187 as shown in FIG. 17B. A thin film of plastic 188 may be attached to the patient's side of the elastic headband to allow disposal of the headband and re-use of the thermistor mounted REM. Thermally conductive jell may be placed in the headband hole to allow better heat transfer from the patient's forehead to the thermistor 183 if thermal conduction is an issue. The wires from the thermistor 183 enter directly into the REM enclosure 184 eliminating the need for an external electrical connector.

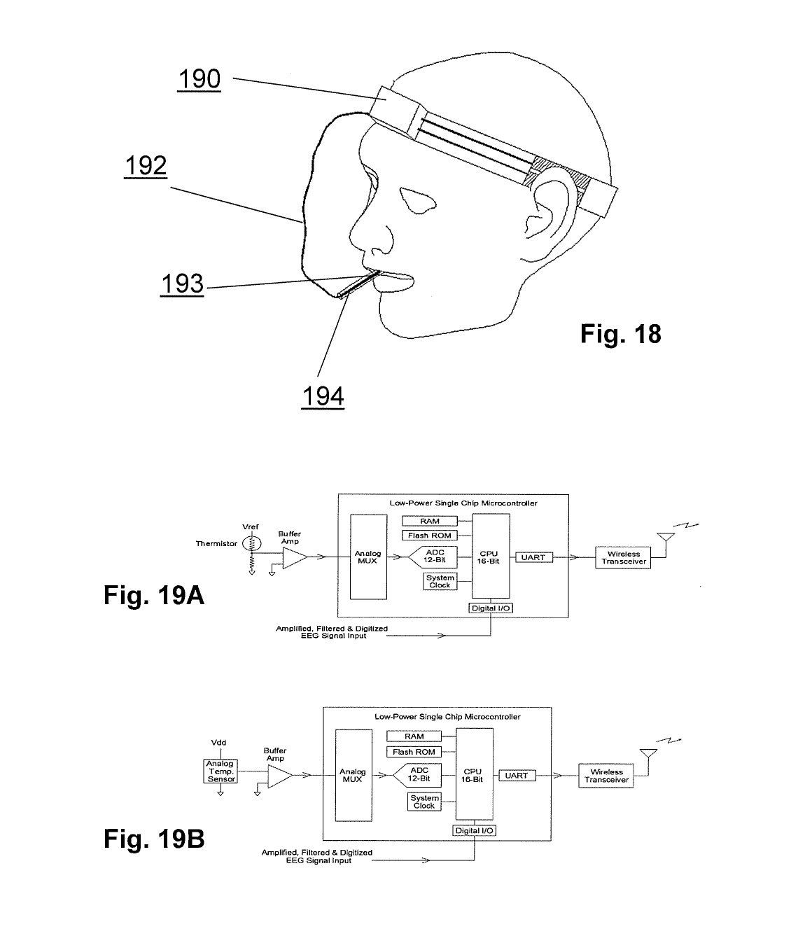

Instead of a forehead temperature measurement, a potentially more accurate temperature measurement may be provided by having a flexible wire 192 with thermistor or digital temperature sensor 193 mounted at the tip of the wire 192, as shown in FIG. 18. A sterile disposable plastic sheath 194 may be placed over the end of the wire 192 and temperature sensor 193. The sensor 193 may then be placed into the patient's mouth for the temperature measurement. The opposite end of the wire 192 enters into the electronic REM enclosure 190 for signal conditioning and acquisition.

To measure the resistance of the thermistor, either a constant current source or constant voltage source is applied to the thermistor. One common method is the use of a constant voltage source applied to the thermistor and a series resistor as shown in FIG. 19A. The voltage drop across the series resistor is amplified and applied to low-power single chip embedded microcontroller (MCU) with integral analog multiplexer (MUX), analog-to-digital converter (ADC), CPU, and universal asynchronous receiver transmitter (UART). A wireless transceiver interfaces to the UART. An example of a possible low-power single chip microcontroller is the Texas Instrument MSP430 connected to a Panasonic CC2560 Bluetooth RF module. This low-power combo is designed for medical applications.

As shown in FIG. 19A, under software control, the MUX connects either the EEG signal or the buffered temperature signal to the analog-to-digital (A/D) converter which is read by the microcontroller (MCU). The microcontroller serially outputs the measurement via the UART to the wireless RF module. The sampled temperature signal is time multiplexed in-between the EEG measurements and sent via a serial data stream over the wireless transmitter to the host receiver MCU (PC, tablet PC, or smartphone). A preamble identifier is included in the transmission to separate the temperature data from the EEG data. Since the thermal response from a thermistor is non-linear, curve fitting or a calibrated lookup table may be used at the MCU host to convert the thermistor's resistance values into calibrated temperature values.

Semiconductor Temperature Sensor with Analog Output Voltage

Semiconductor temperature sensors with a linear analog output voltage are also available for direct interfacing to an A/D converter, as illustrated in FIG. 19B. As known to those skilled in the art, a semiconductor temperature sensor is an IC that combines a temperature-sensing element with signal conditioning, output, and other types of circuitry on one chip. It relies on the change of voltage across a p-n junction in response to a temperature change to determine the ambient temperature. The Microchip MCP9700 is one example. The device requires only a supply voltage and provides a linear 10 mv/C.° analog output voltage.

Digital Temperature Sensor

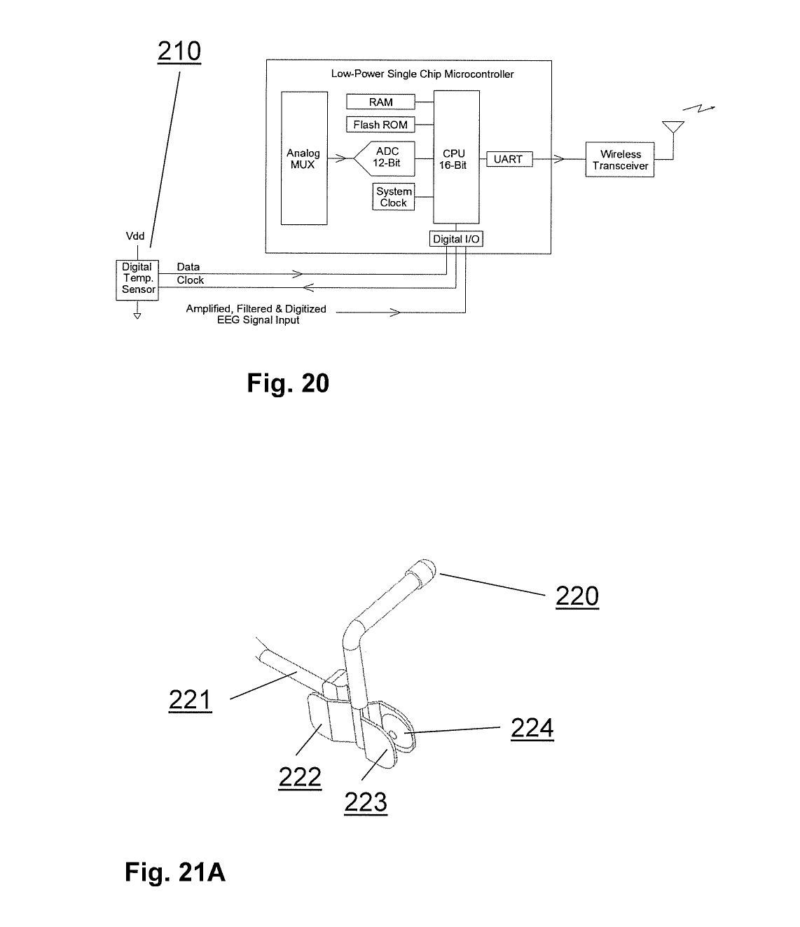

In addition to analog output temperature sensors, a digital output temperature sensor 210 may also be used, as shown in FIG. 20. The mounting of the digital temperature sensor 210 is similar to the thermistor described above. The digital sensor 210 uses a semiconductor to measure temperature and provides a digital serial output for the temperature measurement. The ST STTS751 is one example of a digital temperature sensor 210 that provides a digital serial output interface. The advantage of a digital temperature sensor 210 is it does not require further amplification or an A/D converter. The serial signal attaches directly to a digital input on the embedded MCU. A digital output clock from the embedded MCU sets the serial data transfer rate. Serial values are directly calibrated in degrees C.

Spot Infrared (IR) Temperature Sensor

A fourth method of measuring the patient's temperature uses a miniature spot infrared sensor. The sensor may again be mounted to the exterior of the REM electronic enclosure. The IR sensor then measures the patient's forehead temperature in a non-contact manner through a hole punched in the disposable elastic headband.

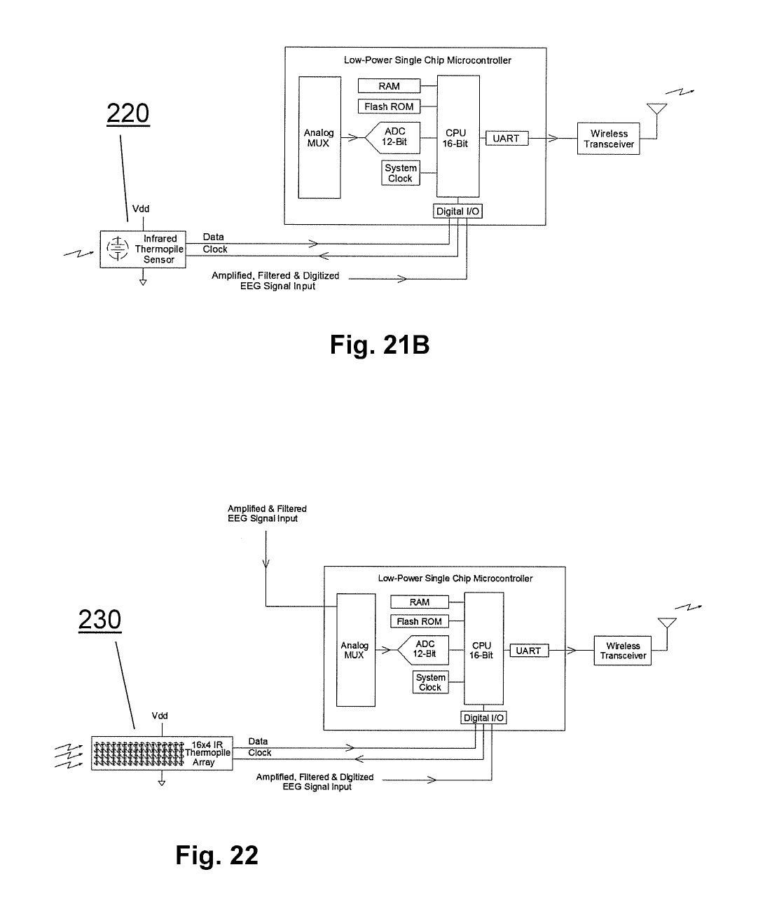

Perhaps more accurately, the IR sensor 220 is also attached to the EEG ear clip 222 and takes a spot temperature measurement looking into the patients ear canal, as shown in FIG. 21A. REF 224 and GND 223 electrodes in addition to Spot IR sensor 220 pass electrical signals back to the REM via cable 221. The Texas Instruments TMP006 is one example of a miniature calibrated infrared spot sensor. FIG. 21B shows the simple digital interface of the infrared thermopile sensor 220 to the embedded MCU within the REM.

Multi-Point Imaging Infrared (IR) Temperature Sensor

Multi-point temperature measurements of the patient's face are also be possible. The Melexis MXL90620 is an example of a 16×4 active pixel thermal array that may be used to thermally image the patient's head. The sensor 230 may be mounted to a stiff wire extending from the REM to allow proper positioning for capture of an IR image of the patient's face. The MXL906520 has a serial interface that is connected to the MCU digital I/O lines, as shown in FIG. 22.

### Accelerometer Based Measurements

Much like described above, another embodiment of the invention incorporates a multi-axis accelerometer and gyrometer into an electronic module. For example, a 3-axis accelerometer and 3-axis gyroscope are mounted onto the biosensor module within an REM and interfaced to the embedded MCU via an UART, SPI or I2C digital serial interface. Alternatively, analog outputs are interfaced to an embedded Analog-to-digital converter (ADC). Popular chips for these functions include various ST Microelectronics ICs such as the LIS 33DL 2823 Accelerometer IC Chip, LIS302DL accelerometer, the LIS331DL accelerometer, and an STMicroelectronics LIS331DL accelerometer with an AKM AK8973 electronic compass. For nine degrees of freedom, one may choose to use an STMicroelectronics LIS331DLH accelerometer and the L3G4200D gyroscope along with an AKM8975 electronic compass, or an L3G4200DH 3-axis digital MEMS gyroscope and a LIS331DLH 3-Axis MEMS accelerometer.

The invention includes the mounting of any one of the various 3, 6 or 9 degree of freedom solutions commercially available with a digital output interface that is captured by the embedded MCU and then stored locally on an SD/microSD card or wirelessly transmitted via Bluetooth RF radio or wired to an MCU via a USB serial interface. The simultaneous recording of the various data streams is kept in place by a real-time operating system environment where time stamps are placed on all samples for ultimate reconstruction in the non-embedded MCU (PC, tablet PC or smartphone).

### Pulse Oximetry Based Measurements

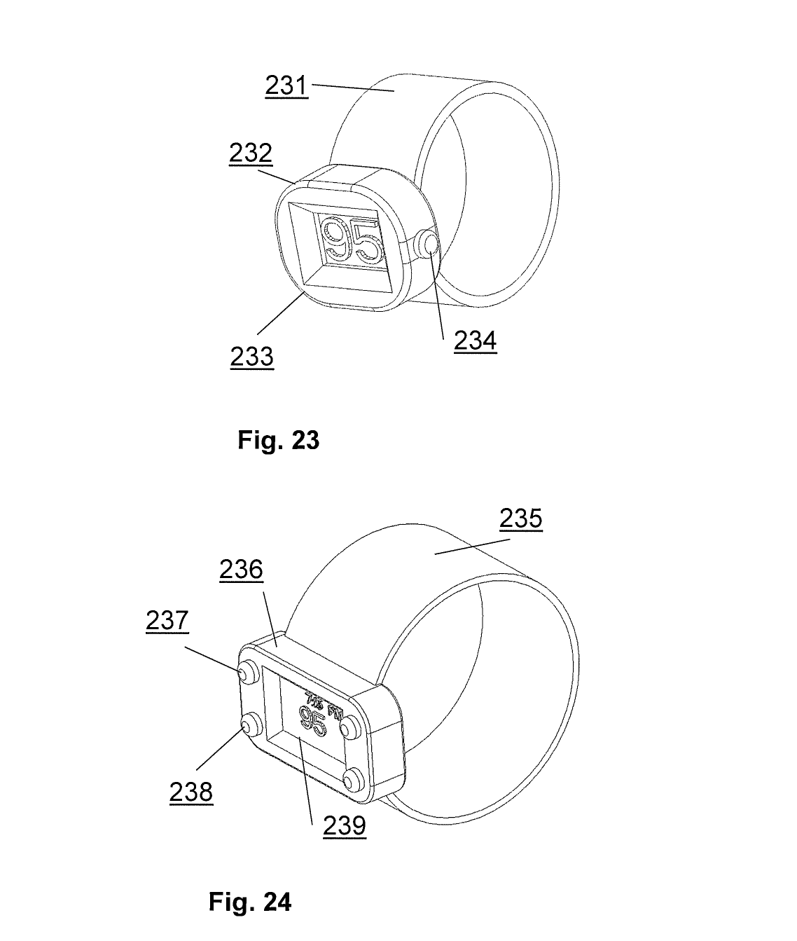

As known to those skilled in the art, a pulse oximeter measures blood oxygenation by sensing the infrared and red-light absorption properties of deoxygenated and oxygenated hemoglobin. As shown in FIG. 23, the oximeter comprises a sensing probe 232 that attaches to a subject's ear lobe, toe, finger or other available body part or surface using a strap 231, for example, and is connected to a data acquisition system 233 for the calculation and display of oxygen saturation level, heart rate and blood flow. Light sources, typically light-emitting diodes (LEDs), shine visible red and infrared light. Deoxygenated hemoglobin allows more infrared light to pass through and absorbs more visible red light. Highly oxygenated hemoglobin allows more visible red light to pass through and absorbs more infrared light. The oximeter senses and calculates the amount of light at those wavelengths proportional to the oxygen saturation (or desaturation) of the hemoglobin. The use of light in the absorbency measurement requires the designer to have a true “light-to-voltage” conversion using current as the input signal. FIG. 23 is a schematic illustration of a peripheral finger mounted REM module, while FIG. 24 is a schematic illustration of a peripheral wrist or ankle mounted REM module including sensing probe 236, strap 235, LEDs 237 and 238, and data acquisition system 239.

Pulse oximeters measure both heart rate and arterial oxygen through either a transmission mode or a reflection mode. Several manufacturers sell OEM modules and designs. Nonin Medical is well known in the space. As well, a lower end embodiment utilizes Texas Instrument's highly integrated MSP430FG437 embedded MCU which reduces the number of external components. The design of a non-invasive optical pulse oximeter using the MSP430FG437 microcontroller (MCU) includes a peripheral probe combined with the embedded MCU either displaying the oxygen saturation and pulse rate on an LCD glass or transmitting the data for recording. In this design, the same sensor is used for heart-rate detection and pulse oximetry. The probe 232 shown in FIG. 23 is placed on a peripheral point of the body, such as a fingertip, an ear lobe or the nose. The probe includes two LEDs 234—one in the visible red spectrum (660 nm) and the other in the infrared spectrum (940 nm). The percentage of oxygen in the body is determined by measuring the intensity from each frequency of light after it is transmitted through the body. Then, the ratio between these two intensities is calculated. Higher quality implementations can utilize TI IVC102a and 102b chips, in combination with an ADS 1255 ADC and MSP430 or digital signal processor. Several designs are available from TI in their health technology product line. Furthermore, TI offers their TMDXMDK08328 Pulse Oximeter PO or SpO2 Analog Front End (AFE) module.

The integration of Pulse Oximeter circuitry into the REM and the attachment of probes to the ear or forehead is a part of the invention for the REM on the head, as well as for the finger, wrist or ankle REM. The combined collection of heart rate, blood oxygen in combination with EEG brainwave data is a unique aspect of the invention.

### Galvanic Skin Response or Electrodermal Response

Galvanic Skin Response (GSR) or Electrodermal Response (EDR) is the changing of electrical skin resistance due to psychological condition. The change is caused by the degree to which a person's sweat glands are active. Psychological stress tends to make the glands more active and this lowers the skin's resistance (typically measured as micro-Siemens). Common designs sample at 10 Hz across two electrodes. To measure Skin Conductance (SC), a very small voltage is applied across these electrodes (0.5V). By measuring the current that flows, conductance can be measured. By Ohms law, Resistance=Voltage divided by Current, therefore Conductance=Current divided by voltage, the reciprocal of resistance. The unit of resistance is the Ohm, and Conductance used to be expressed as Mho, but the preferred unit of conductance is microSiemens. It is the reciprocal of MegOhm. Zero resistance (a short circuit) is infinite conductance, 1 MegOhm is 1 microSiemens, 2 MegOhms is 0.5 microSiemens, 100 kOhms is 10 microSiemens, and so on.

In the invention, one could choose to place two additional electrodes on the REM inner surface and allow the skin conductance between them to be measured in the vicinity of the EEG sensor, or more interestingly, the two electrodes on the ear could be multiplexed at 10 Hz when not used for the EEG. If necessary, four electrodes could be placed on one ear or two electrodes on one ear could be used for REF and GND for the EEG while the two different electrodes could be placed on the opposite ear for simultaneous use in making contemporaneous GSR measurements. In one exemplary embodiment, EEG, GSR, pulse oximetry (for heart rate and arterial oxygen), temperature, and accelerometer based data streams are all collected by the head based REM.

### Cerebral Blood Perfusion and Vaso-Motor Reactivity

Cerebral Blood Perfusion (CBP) or other means to assess the vasculature of the brain can be used as additional biosensor data streams. For instance, a tiny microphone temporarily inserted within the ear canal of a subject can record the minute auditory sounds emitted by the circulation and perfusion of blood through the brain based vasculature either (i) when the subject is at rest or (ii) during activated tasks such as (a) hyperventilating, (b) breathing C0₂, or (c) breathing enhanced purity oxygen of greater than 21% content relative to dry air. This passive microphone recording can be sampled with high sample rate, for instance from 8,000 samples/sec to over 50,000 samples/sec with a high precision analog-to-digital converter (ADC) such that the 16-bit or 24-bit digital output is transferred via wire or wirelessly to an REM attached to the body. Recordings can vary in length and be taken while the subject engages in standard cognitive and sensory tasks.

These bio-signals can then be signal pre-processed and then signal processed for cerebral blood perfusion differences associated with a disease state or injured condition. For instance, these passive microphone based measurements and sounds could be analyzed to detect injury to the vasculature in the case of a concussion or traumatic brain injury. In the alternative, these passive defects in blood flow sound could be used to differentiate either alone or in a multi-variate statistical predictive model a neurodegenerating brain such as from someone with Alzheimer's disease, Parkinson's disease or other neurological brain related disease, injury or condition (such as migraine or neuropathic pain). The present invention and its use in neuropsychiatry and mental illness is equally fortuitous as one can imagine Cerebral Blood Perfusion based differences in depression, bipolar disorder, schizophrenia, anxiety disorders and other mental illness based psychosis.

A minor modification of the present embodiment could be used to then measure the “vaso-motor reactivity” (VMR) of a human or animal subject. Poor VMR would then possibly be an indication of increased risk of death, TIA or stroke. An example protocol to measure VMR would consist of a 2 minute period of deep breathing or hyperventilation through the mouth. One could investigate the impairment of VMR in brain related injury and disease states. If it was observed, it would unfortunately provide evidence for marked hemodynamic changes within a subject.

A non-limiting illustrative protocol would consist of 1 minute of passive ear canal microphone recording to assess the CBP. Then ask the subject to hyperventilate through their mouth for 2 minutes, where one then records again the next or fourth minute of the protocol while the patient continues to hyperventilate as an assessment of VMR. Since it is known that the EEG shift associated with hyperventilation is that the amplitude of EEG goes up while the peak frequency goes down, this embodiment could be a useful means to assess both.

### Peripheral Electronic Modules Gather Limb Data in Addition to the Head Based REM

The invention also includes the use of peripheral electronic modules to collect limb data, either or both arm or leg at the hand/wrist or the ankle/foot, at the same time that the head REM is collecting brain/skull related biological signal data. For instance, while a human subject is undergoing a vestibular or balanced based assessment during a concussion battery of tests, the human subject could be asked to stand on a firm surface in various postures, consistent with the Balanced Error Scoring System or BESS. Rather than have an athletic trainer or manager subjectively score and evaluate the human subject for various subjective errors, as is presently done, a multi-axis accelerometer can measure objective biological signals of the human subject's stability based on their head movement and motion while conducting the task and while the EEG sensor is collecting contemporaneous brainwave data. Similar accelerometer and position/motion sensors place near the hand and/or foot further capture extremity motion and assess with objective data the human subject's ability to react to change when asked to stand on an elastic or unstable surface while accelerometers and gyrometers in the head REM continue to measure head/trunk stability. In one embodiment, additional accelerometer data is collected by a limb REM attached to the hand or wrist while a third REM, attached near an ankle, further quantifies the human subject's balance skills simply, quantitatively and inexpensively using a 3, 6 or 9 degree of freedom based system at each physical location (head, hand, foot). In addition to conducting these balance related tasks on a firm surface, use of an inflatable and disposable pillow or air cushion made from strong plastic provides an inexpensive means to assess the human subject on a pristine and unused soft and unstable elastic surface. When reusable foam cushions are permissible, like the Airex model recommended in the BESS instructions, they are excellent second surfaces for A versus B comparisons. In instances where repeated use by multiple human subjects is not permitted, such as in medical evaluations and assessments, the use of a compact, disposable, and inexpensive elastic and unstable inflatable pillow device for a human subject to stand on could advantageously assist in a concussion or other balance/vestibular system assessment and is a part of the invention. Here, the same A versus B comparison is possible, but with the added benefit of a single use disposable unstable surface.

Mobile Peripheral MCUs as a Peripheral REM to Record from Built in Sensors

It may be preferable in the invention to utilize the built in sensors in commercial MCU devices including laptop PCs, tablet PCs and smartphones in addition to or in substitution of a wrist based REM. In particular, most mobile MCUs have some assortment of the following built in MCU sensors:

1. keyboard/mouse or touch screen,

2. microphone,

3. accelerometers,

4. camera or eye tracking biosensor,

5. temperature,

6. magnetic compass,

7. GPS global positioning system.

As a non-limiting example, recording neuropsychological data occurs via the keyboard key strokes, mouse clicks or touchscreen panel events, where each provides a 3 dimensional vector for each event (x, y, t), where x,y are the coordinates of the location on the screen where the event occurred (most typically indicating correct or incorrect) and t is the time of the event relative to some internal master experiment clock, often the system clock of the peripheral MCU but perhaps a faster real-time clock built into the Bluetooth radio more advanced v3.0 and v4.0 protocols. Thus, mouse clicks, keyboard strokes or touch panel events are more or less equivalent, as long as they are compared to themselves in each instance. Additional data is derivable from the previous instances of the cursor or finger in that direction and velocity information can be inferred by looking back a few clock cycles to determine the derivative of the position or velocity (both speed and direction). This data stream provides the equivalent of many neuropsychological tests conducted today, including data comparable to the CogState cognition battery, the ImPACT test, the CANTAB battery, and other similar computer delivered neuro cognitive assessment tests.

Moreover, most of these MCU devices have a sound card for presentation of auditory stimuli but also have a microphone to record the voice and responses of the human subject during verbal related tasks and stimulations. Thus, an 8-bit 8 kSam/sec microphone recording can serve as a base or minimum level of data, while a 16-bit 16 or 22 kSam/sec recording provides a higher fidelity data stream at increased data transport constraints. For instance, during the PASAT task, the recording from the microphone may be used for automated scoring, reaction time information and other signal processing features to be extracted later in time off-line. In either case, the recording of the microphone provides a convenient second built in sensor data stream for comprehensive analysis of the human subject.

Built-in accelerometers (which often include gyrometers and magnetic compass sensors) enable the objective recording of motion sensing activities that are both intentional and unintentional while the MCU is being held by the human subject. The use of the peripheral MCU accelerometers are of particular interest to replace a wrist based REM and use the built-in sensors of the peripheral MCU instead in order to simplify the overall multi-modal data acquisition system. For instance, a single head based REM is used in conjunction with motion based accelerometer measurements made in the peripheral MCU of tablet PC while the subject is asked to conduct prescribed movement tasks holding the tablet PC. Risk of damage would be an issue but secure tethering via glove or Velcro closure to a Velcro glove mitigates risk of tablet or smartphone damage. In particular, a resting state assessment of stability could be made as determined by the RMS deviation of position or the standard deviation of the vibrational noise collected while putatively at rest.

Another non-limiting embodiment of the invention includes measures of dynamic stability. A task is assigned to a human subject under evaluation to step laterally up and over an obstacle several times from left to right while facing forward. This sort of dynamic stability task then assesses aspects of dynamic stability not picked up by static or resting stability. It could in fact be the case that resting state stability or dynamic state stability measurements or other objective features derived from the built-in accelerometers would provide important diagnostic features in the development of multi-modal signatures of brain health, disease and injury.

There is often a front facing camera in conventional peripheral MCU devices. Laptops typically have web cameras; tablet PCs and smart phones typically have front facing cameras for video conferencing applications popular over the internet. These video cameras provide a wonderful built-in sensor to capture important streams of biological data from an individual. In one embodiment of the invention, random photographs of the face of the subject are taken intermittently while the human subject conducts a series of various tasks in order to provide positive biometric identification of the subject conducting the task, even if conducting the task remotely or in the privacy of their own home. Especially if the images are sampled randomly or with sufficient frequency to insure consistent use by a single human subject, this approach to unique biometric identification prevents test fraud or misuse in a home assessment tool to insure who was actually taking the test. Also, it is well known that eye tracking or saccade movements are very informative regarding neuro-ocular motion. Image movies captured at video 30 Hz sample rates of the eyes and face of a subject conducting an assessment task could be stored and analyzed later independently or in combination with other sensor data. Also, as known to those skilled in the art, other types of biosensors may also be used to track eye movements by measuring the positions of the eyes with respect to time.

As a non-limiting example of this, this combination of video and EEG is commonly employed in seizure detection and epilepsy diagnosis to the extent that it is more possible. This is commonly done with EEG already in expensive and cumbersome Video-EEG systems. Thus, the use of a tablet PC or smartphone as the video portion of a portable ambulatory video-EEG provides a much less expensive system. This ambulatory video-EEG system would further have the advantage of the other built-in sensors present in the peripheral MCU on which the data is being recorded and stored. This includes the ability to record voice messages and events verbally in a journal, rather than write things down, including motion sensing via accelerometers if a seizure were to take place while holding the peripheral MCU and could be correlated in time and with head based REM measures of brainwave activity, motion of a seizure, peripheral blood and oxygen if a pulse-oximeter is in the head REM and even temperature if built in to the head REM.

Temperature may be monitored by the peripheral MCU, not as a record of the individual human subject's temperature but rather as an objective record of the environmental temperatures that the subject was moving through to enable trigger based analysis in the case of seizure detection or other monitoring based investigations.

Lastly, Global Positioning System GPS based measurements of location may be used to construct motion maps on a larger scale to complement the detailed accelerometer based measurements to again provide detailed history to enable trigger based analysis in long term monitoring assessments for problematic brain based activity such as seizure detection and epilepsy based diagnosis.

### Embodiments Around Activated Patient Sensory and Cognitive Stimulation

Application of sensory stimulants to the patient allows more focused and detailed evaluation of multiple modes of biological signal data streams. Multi-modal data can be acquired by measuring EEG signals at the same time that accelerometer based signals, temperature signals, and other biological signals are being simultaneously acquired before, during and/or after a patent's response to a sensory stimulant or cognitive challenge.

Visual Stimulation

Visual stimulants such as photic stimulation while a subject's eyes are closed or via the presentation of certain types of affective photographic images can be utilized either independently or via the data capture microprocessor device (MCU) (computer, tablet PC, cell phone, or other dedicated custom device with microprocessor and wireless connectivity) used to collect the wireless bio-signal data from the various REM units on the head, hand/wrist or foot/ankle. Various graphics assessments are displayed on the data capture display in which the patient can respond as well via touch screen, voice, motion, mouse clicks and keyboard strokes. In principle, newer user inputs such as particular brainwave patterns and accelerometer based signatures (encryption of passwords in precise accelerometer based movements for instance) are also part of the invention.

Auditory Stimulation

Sensory stimulants such as sound also may be provided either independently or with the data capture microprocessor device (MCU) (computer, tablet PC, cell phone, or other dedicated custom device with microprocessor and wireless connectivity) used to collect the wireless bio-signal data from the REM. Sound events are triggered via the speaker or sound card on the computer at various times for the patient to respond to both instructions as well as auditory stimulations of a novel nature as described elsewhere. This may be through the speakers as well as through ear buds or other personalized listening devices.

Gastronomic Stimulation of the Taste and Gastrointestinal Tract

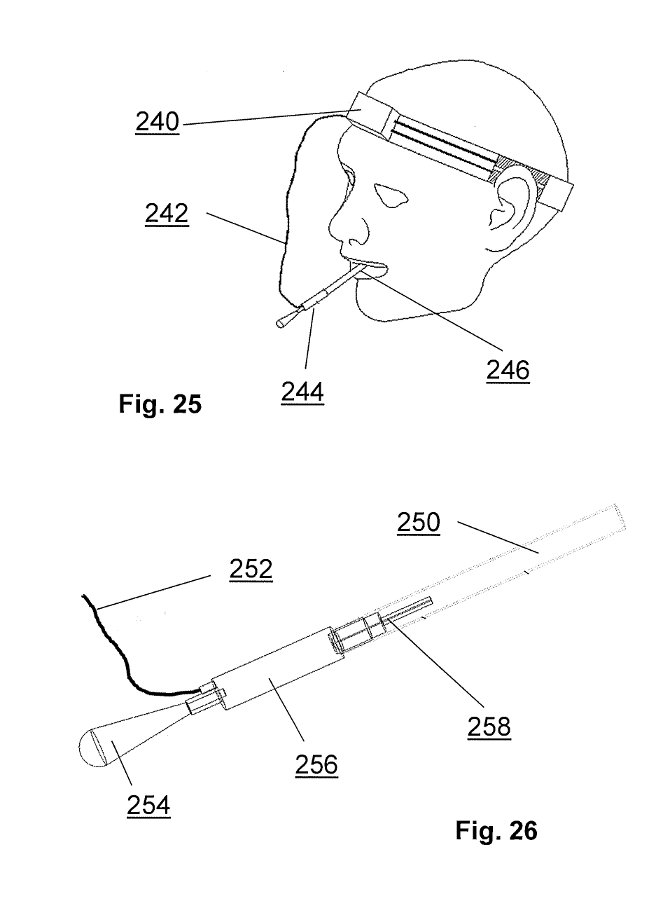

Besides visual and auditory sensory stimulates, gastronomic stimulation is also possible with the invention. In one embodiment, as shown in FIG. 25, a miniature fluid dispensing apparatus 244 is inserted into the patient's mouth controlled via a hardwired connection 242 to the REM 240. Under software control, the REM 240 would inject a small volume of liquid stimulant into the patients' mouth via a disposable straw 246 at an appropriate time.

FIG. 26 shows one example of a fluid dispensing apparatus that generates jets of a liquid stimulant to the patient's mouth. The liquid stimulant is contained inside a small elastomer bulb 254. The stimulant fills the bulb cavity and stretches the elastomer bulb creating a positive pressure. An optional method would be to use a rigid cavity for the liquid stimulant with a portion of the vessel pressurized with an inert gas. The elastomer bulb or pressurized vessel is connected to a high speed gated solenoid valve 256. The output port on the valve 256 attaches to an orifice dispensing nozzle 258. A disposable plastic straw 250 is attached to the nozzle 258 and is placed into the patient's mouth.

Upon an appropriate command from the data acquisition computer or MCU, the REM generates a short duration digital output, transmitted through electrical connection 252 between the REM and the gastronomic delivery device. The digital output gates the high speed solenoid valve 256 open for a short duration. The pressurized fluid would pass through the solenoid valve 256 and nozzle 258 thereby ejecting micro-drops of fluid or particles down the length of the disposable straw 250 and into the patient's mouth. The pulse width applied to the solenoid valve 256 determines the volume of fluid dispensed.

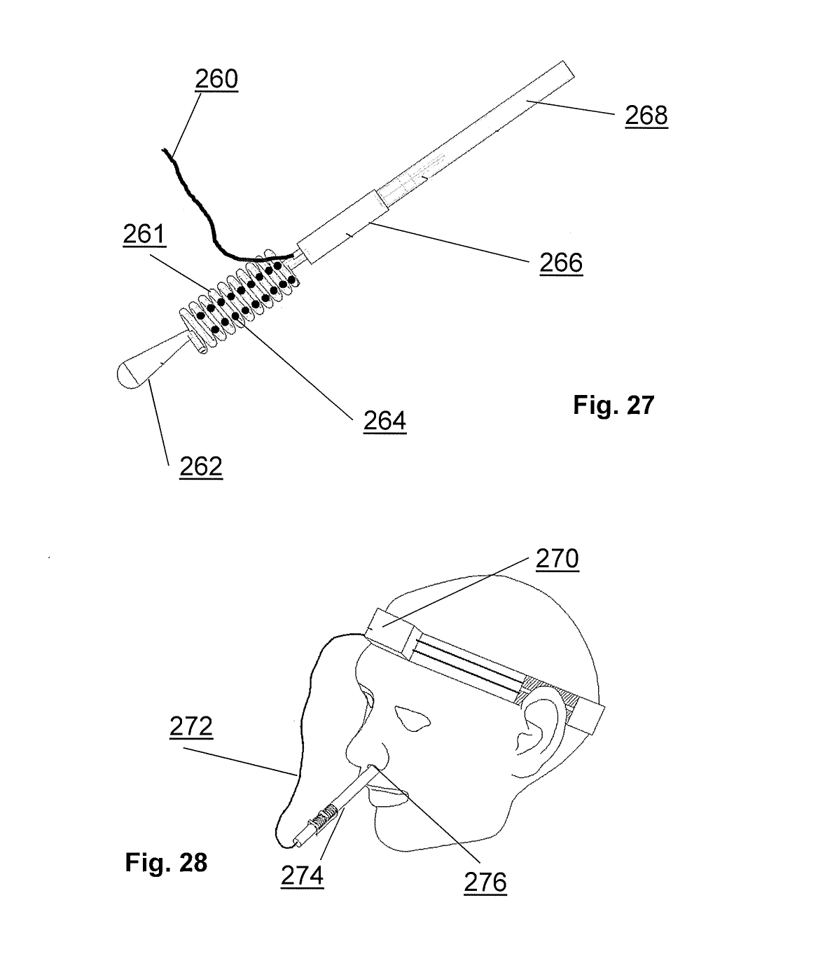

In the above example, only one type of stimulate can be utilized. FIG. 27 shows a method of having a variety of stimulants available. Drops of the different stimulants are contained inside a coiled piece of tubing 261. Gas air bubbles 264 separate each fluid bolus. The prefilled coil contains a pattern of desired stimulants (i.e. liquid A, B, C, etc.). One end of the prefilled tubing 261 connects to the inlet of the gated solenoid 266, and the opposite end connects to a pressurized air source 262, either a gas filled elastomer bulb or pressurized container. Dispensing is controlled via digital pulses applied through the electrical connection 260 to the REM opening/closing the micro-solenoid valve dispensing the stimulant bolus through the dispensing or atomizing nozzle 266 down the disposable straw 268 and into the patient's mouth until an air bubble is reached. An optical bubble sensor (not shown) may be used to sense the air bubble separator between the different stimulants.

Olfactory Simulation

A simple means of olfactory stimulation could be as simple as using an UPSIT card or cards from Sensonics for the University of Penn Smell Identification Test (UPSIT) to provide olfactory stimulation to the nose of an individual at pre-defined times indicated by the instructions provided by the peripheral MCU software. This could include scratching and sniffing each of any number of cards with odors as prescribed and directed. The results are automatically recorded by the various multi-modal biological sensor data streams being generated from the human subject under assessment at that time.

In a more automated fashion, application of a gaseous stimulant into the patients' nose 276 at an appropriate time is also possible using a digital output sent down interface cable 272 from the REM controller 270 to a gas dispensing apparatus 274, as shown in FIG. 28. The same apparatus shown in FIGS. 26 and 27 could be used with the orifice dispensing nozzle replaced with an atomizing nozzle. The atomized vapor would pass down the disposable straw into the patient's nose.

Another automated method to generate an olfactory stimulant uses “scratch and sniff” materials, but rather than on individual manual cards, automated by the REM and MCU system. For instance, as a non-limiting example, different scratch and sniff stimulants are deposited and dried onto different portions of a small threaded lead-screw 286 of FIG. 29. A micro-motor 284 is attached to one end of the lead-screw. The micro-motor 284 is controlled via a digital output from the REM delivered through electrical cable 282. As the lead-screw 286 rotates, a follower nut 280 traverses down the lead-screw 286 scratching the different stimulants. The odor would propagate down the disposable straw 288 to the patient's nose.

Combined Physical Motion and Cognitive Challenges

A simple means of challenging not only the cognitive status of an individual, but also their ability to exhibit both fine and gross motor control are an integral part of the invention. In particular, a pre-defined path of motions of the head REM, hand/wrist REM and/or foot/ankle REM are presented visually on the MCU display screen and the voice instructions instruct the subject. Alternatively, a short demonstration movie could be shown exemplifying the motion that the subject is to undertake. Then, when prompted to begin, the subject would need to remember the sequence of physical maneuvers and execute the task while the head, wrist and ankle based REMs record the motion of the human subject through their built in 3, 6 or 9 degrees of freedom based accelerometers, gyrometers and magnetic compass sensors collectively referred to as accelerometers but meant to also include gyrometers and magnetic compass sensors for either 3, 6 or 9 degrees of freedom.

As a non-limiting example, a human subject could be asked to stand on the floor in the center of an exam room, bend over to pick up a sheet of paper off the floor, turn to their right 90 degrees, and then extend their hands to place the paper on a nearby table, similar in nature to one of the tasks in the Mini-Mental State Exam. Accelerometers could track either the head alone, or the head in combination with the wrist or the head in combination with both the wrist and the ankle to create a quantitative motion profile the subject. Healthy controls could be assessed and normative data produced for cross-sectional diagnostic assessments. When possible, baselines could be collected for within subject baseline adjusted assessment longitudinally over time or after putative events such as concussive impact, chemotherapy induced cognitive impairment, or other unexplained need for within subject assessment of change.

In another non-limiting example, a series of instructions could be given by the data gathering MCU (PC, tablet PC or smartphone) and the subject asked to follow the auditory instructions step by step. As they do so, the motion based accelerometers are recording the quality of their performance to conduct the tasks properly.

### A System for the Collection of Multiple Streams of Brain Health Assessment Data

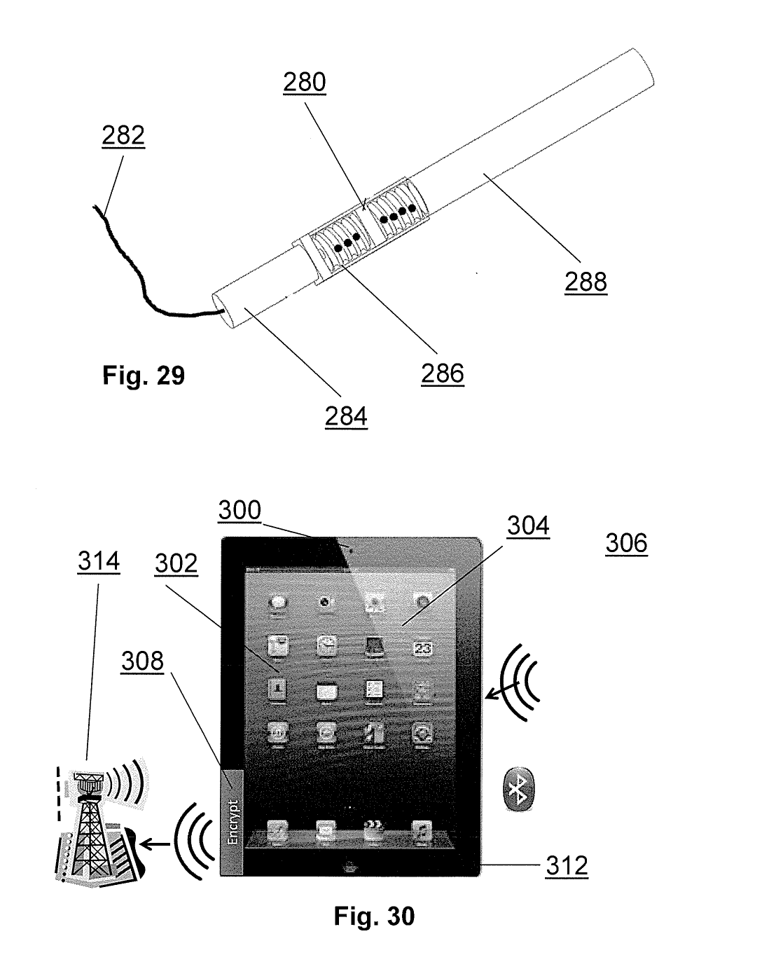

Another embodiment of the invention includes a data recording and analysis system that includes at least one REM placed on the head of a human subject to record brain related biological health signals, a peripheral MCU, and a cloud based enterprise information technology infrastructure to process and report the data that has been collected. In particular, FIG. 30 illustrates an electronic REM module 306 on a subject's head transmitting wireless data to peripheral MCU (in the form of a tablet PC) 304. While the data is being collected through the Bluetooth port in the MCU, the camera 300 is recording a movie of images of the subject as they perform tasks to not only verify their identity but also to analyze their eye and facial movement for features of interest (including saccade). Microphone 312 records the voice of the subject for voice recognition analysis, while accelerometer and gyrometer 302 measure the stability or lack thereof of the subject, while touch screen 304 of the peripheral MCU records events at precise times and spatial (x,y) locations on the touch screen. Finally, when all the various data streams are complete, along with demographic and personal health information, the entire package of information is encrypted locally using AES-128 or AES-256 bit encryption 308 before being transmitted to the virtual or remote based servers through an internet connection 314 which could be Wi-Fi, Ethernet, cellular or satellite in nature.

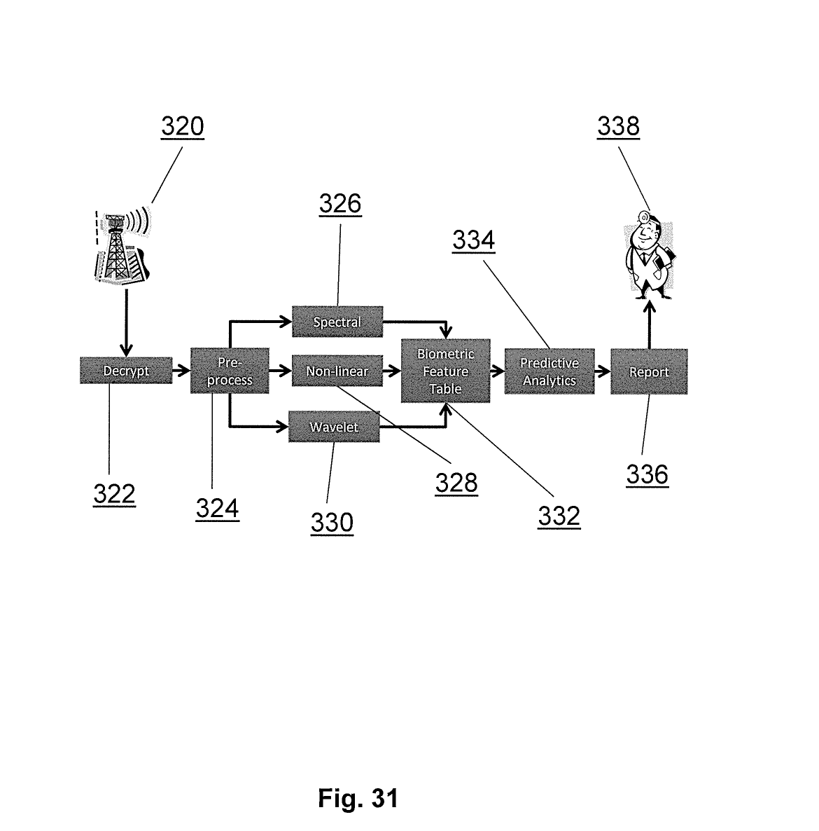

Once the data is received by the virtual server 320 connections, as shown in FIG. 31, it is decrypted at 322 by appropriate algorithms with the key and then sent on for pre-processing to identify areas of artifact such as eye blink, drop outs, saturated rails, movement artifacts, EKG artifacts or other known artifacts at 324. Once the artifacts have been identified and characterized, the regions of good data for each of the various data streams are passed through signal processing software to extract candidate features from each of the data streams available. In particular, a spectral analysis or FFT module 326 is applied to the data signals, a non-linear dynamics module 328 is applied, as is a wavelet transform module 330. Once each module has extracted the relevant and candidate features from each block of data, the software then assembles an extracted biometric feature table 332 including each of the candidate features from each of the streams of data, including a listing of the artifact features as possible diagnostic features as well. From the biometric feature table 332, predictive analytics 334 are run on the unknown subject and the predictive models generate an output by either classifying the subject into one of several groups or classes or alternatively predicts a regression score as an output. These information are then compared to either baseline/earlier data from that same subject or from a demographically match population's normative data and a report 336 is generated. The report 336 is then sent electronically to physician 338 who is able to remotely interpret the report and provide their interpretation before the report is sent back to the point of care for action by the healthcare provider who captured the data in the first place.

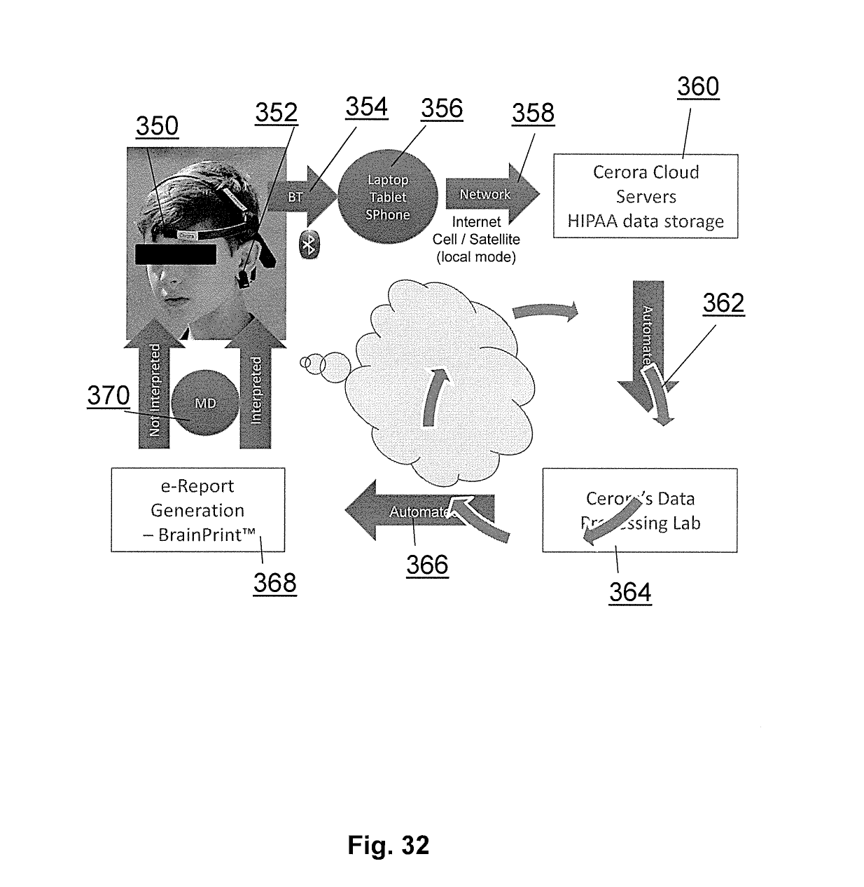

An alternate view is provided by FIG. 32 where active sensor electronic module 350 is mounted with ear clip 352 on the human subject's head. The Bluetooth or other local means of connectivity 354 transfers the data to the peripheral MCU 356 (laptop, tablet or smartphone) whereby the data is encrypted and sent to the network 358 via internet, cellular or satellite. Once at the virtual and remote servers 360, the data is automatically decrypted and processed 362 at the data processing center 364 remotely. Once pre-processing, signal analysis, and predictive modeling are complete, the system automatically 366 generates a report 368. This report is then sent back to the point of care if requested by an appropriate physician 370 or to an appropriate physician 370 for interpretation before being sent back to the point of care to insure that a physician remains a part of the diagnostic cycle.

### Accelerometer Measurements to Quantify Motion, Balance and Gait

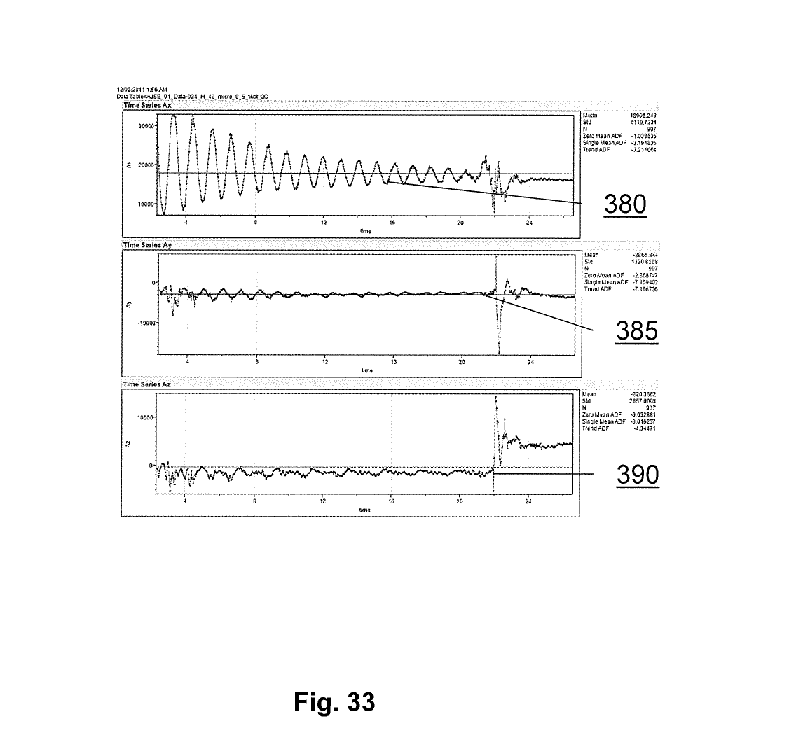

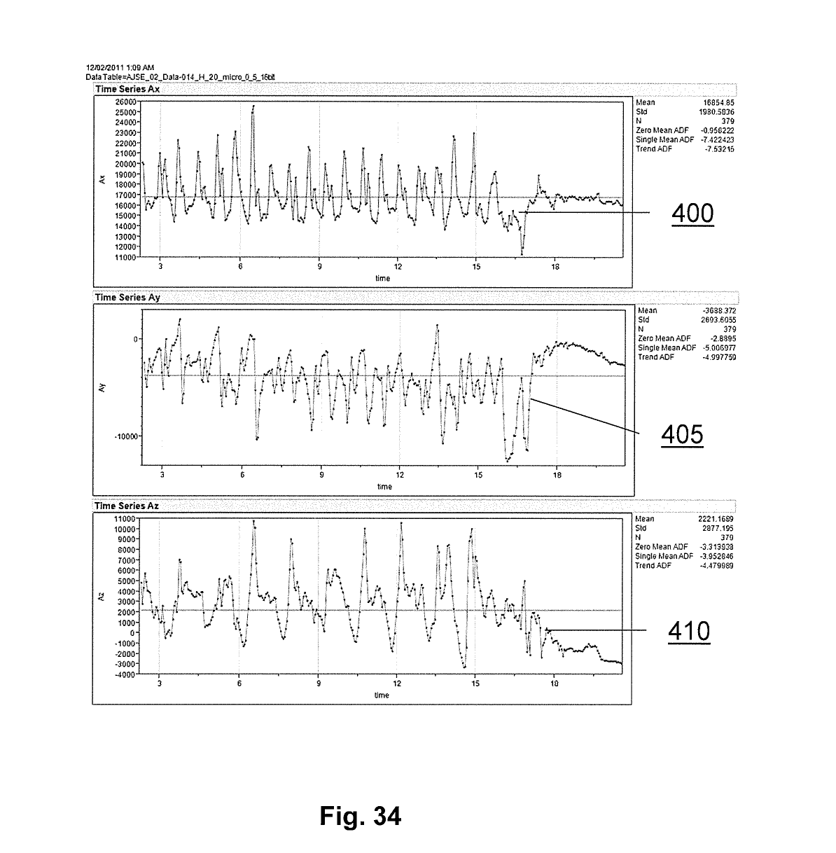

Another embodiment of the invention includes means for collecting multi-axis accelerometer measurements. FIG. 33 illustrates three traces collected in a single 3-axis MEMS accelerometer used as a pendulum to calibrate the device. Trace 380 shows a decaying sinusoidal oscillation in the x-axis, while traces 385 for the y-axis and 390 for the z-axis show little to no oscillation. Through a calibration procedure like this or through the use of a vibrating plate generating a known frequency of oscillation, one can calibrate an accelerometer based motion detection apparatus. In FIG. 34, one can see data collected from a human subject who was wearing the 3-axis accelerometer on his wrist as he swung his arm back and forth while walking through an obstacle course in his laboratory. Trace 400 shows the x-axis, 405 shows the y-axis and trace 410 shows the z-axis. Time runs along the x-axis and the acceleration in each direction is plotted along the y-axis of each trace.

### Use of the Multi-Modal System to Create Multi-Modal Signatures for Disease or Injury

Using the system of the invention, one can build extracted biometric tables that include features extracted from multiple modes of biological signal data. As a non-limiting example, two groups of subjects, group A who experienced a concussion (mTBI) or mild traumatic brain injury, and group B who did not and serve as Controls (CTL), were recruited under the supervision of an Institutional Review Board. Participants from both groups A and B were scanned identically with an electronic REM module including a single electrode EEG. A 5 minute protocol was implemented including 30 seconds Eyes Closed, 30 seconds Eyes Open, conducting the King-Devick test for approximately 3 minutes and then 30 seconds Eyes Closed, 30 seconds Eyes Open again. The stop watch times and errors for each card of the King-Devick test were recorded manually by the test administrator while the peripheral MCU (a laptop computer) presented the cards and recorded the responses of the individuals via the microphone. The data was blinded to participant for the purposes of artifact detection, signal processing and feature extraction. The extracted feature data table was then quality controlled and scrubbed to remove as many errors as possible. The total time for the King-Devick test was created as one extracted variable and underwent a logistic classification model. The result of this model indicated that the King-Devick time alone predicted the classification of the individuals approximately 62% of the time. Independently, the relative power in each of the delta, theta, alpha, beta and gamma bands was analyzed in a logistic classification model where the EEG feature was the predictor x-variable and the clinical outcome (grp A or B) was the outcome y-variable in the model. The analysis was conducted in JMP Pro v10 from SAS (Cary, N.C.).

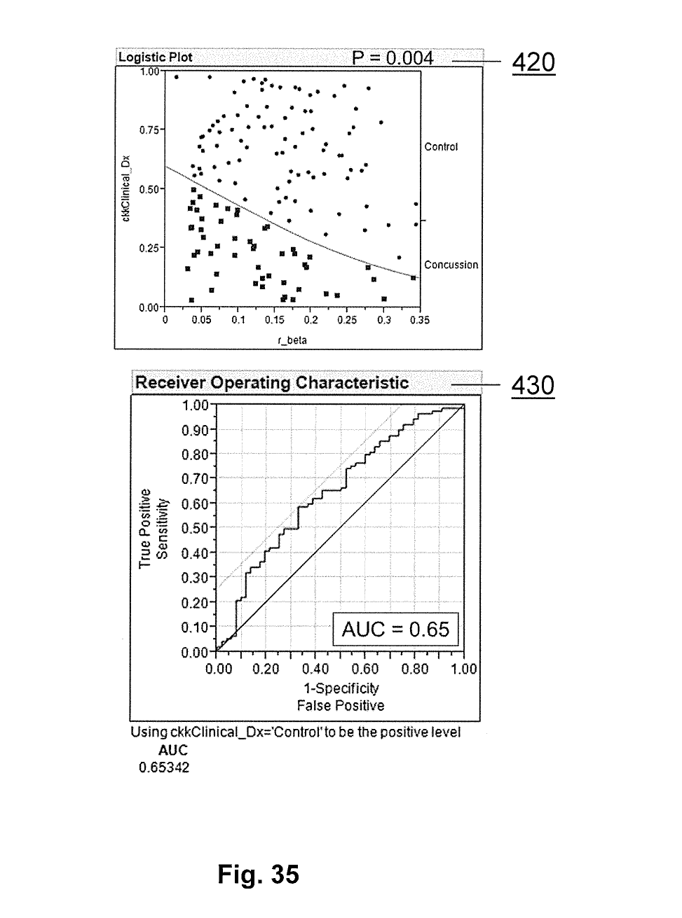

FIG. 35 illustrates the logistic plot 420 for the relative-beta power (from 12-30 Hz) showing a decreased relative beta power in the concussed group A relative to control group B. When one constructs the receiver operating characteristic (ROC) curve 430, one can see that the EEG feature alone predicts with accuracy approximately 65% of the time as defined by the summary AUC statistic.

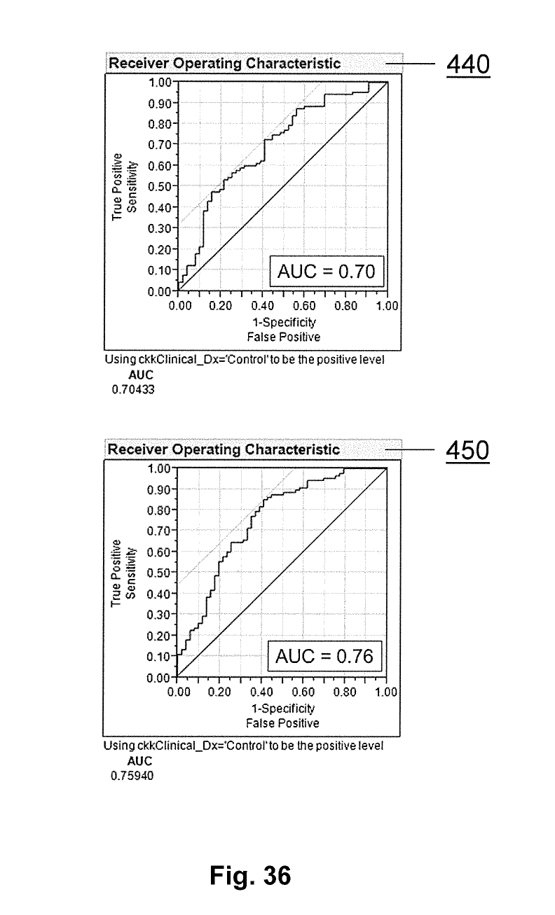

FIG. 36 illustrates in ROC plot 440 that the area under the curve (AUC) is now 70% when the King-Devick test time (a cognitive measure of the subjects brain) is combined with the relative beta EEG power (a brainwave measure), creating a multi-modal signature. When one adds the co-variates of age and gender, the AUC raises to 76% as shown in ROC plot 450, fully corroborating the system and methods of the invention.

The following tables Table 1 through Table 4 list the statistically different features found between the concussed subjects and the non-injured control subjects in either the Eyes Closed or Eyes Open state by either a parametric Analysis of Variation (ANOVA) which is equivalent to the Student's t-Test for two groups or the Wilcoxin non-parametric test which does not rely on normalcy of underlying distributions. In each case, the features have been sorted by most significant (smallest False Positive Rate p-value) at the top to least significant (largest FPR p-value) but only including those features where FPR p-value p<0.05, consistent with the community norm. The extracted features either come from the EEG brainwave sensor in which case the feature name begins with either “Relative_P” or “Absolute_P” which indicates the relative power or absolute power, respectively, within the power spectrum after Fast Fourier Transform of the raw time series information so that Relative_P4_{—}6 would mean the relative power in the 4 to 6 Hz band and Absolute_P34_{—}36 would mean the Absolute power in the 34 to 36 Hz band. Delta is from 1-4 Hz, theta from 4-8 Hz, alpha from 8-12 Hz, beta from 12-20 Hz and gamma from 20-50 Hz in this study. In addition, the mean frequency of the distribution Mean_F, standard deviation of the distribution of frequencies Std_F, skewness Skew_F and kurtosis Kurtosis_F were all calculated along with the peak frequency with the most power Peak_F. In addition, the neuropsychological testing performance characteristics from each of the three King-Devick opthalmological saccade cards C1, C2 and C3 in either round one R1 or R2 are noted within the feature name. Times are indicated as secs at the end, errors are indicated as Errs. The total time for a round of cards would be shortened to KD_R1_Tsecs, while the final time of the whole test taking the fastest time with the least amount of errors from the two rounds through the cards was designated KD_Fsecs or KD_Final_secs in time or KD_Ferrs in errors. In some instance, power was either added, designated for instance by Relative_alpha+beta, divided in the exemplary case of Relative_theta/Relative_beta or the combination, as exemplified in the case of Relative_alpha+beta/Relative delta+theta.

Table 1 illustrates significantly different features (sorted from most significant to least significant) between concussed and control subjects during the Eyes Closed task as determined by Analysis of Variation (ANOVA) or equivalently Student's t-test for two groups (in JMP Pro by SAS). Features only listed for those with False Positive Rate p-value p<0.05.

TABLE 1

| | ANOVA |
|---|---|
| Eyes Closed | FPR p-value |
| Extracted Feature | Prob > F |
| KD_R1_C1_Secs | 0.0001 |
| Relative_P18_20 | 0.0002 |
| KD_R1_Tsecs | 0.0003 |
| KD_R1_C3_Secs | 0.0004 |
| KD_R1_C2_Secs | 0.0013 |
| Relative_P12_20 | 0.0014 |
| Relative_P20_22 | 0.0015 |
| KD_Final_secs | 0.0015 |
| Absolute_P2.5_4 | 0.0024 |
| KD_R2_C1_Secs | 0.0029 |
| KD_R2_Tsecs | 0.0030 |
| Relative_P14_16 | 0.0033 |
| KD_R2_C3_Secs | 0.0035 |
| Absolute_delta | 0.0042 |
| Relative beta | 0.0045 |
| Relative_P34_36 | 0.0047 |
| Absolute_P8_10 | 0.0049 |
| Relative_P12_14 | 0.0054 |
| Relative_theta/Relative_beta | 0.0066 |
| Absolute_theta/Absolute_beta | 0.0066 |
| StdDev_F | 0.0066 |
| KD_R2_C2_Secs | 0.0081 |
| Relative_P8_10 | 0.0116 |
| Relative_P32_34 | 0.0121 |
| Absolute_P4_6 | 0.0161 |
| Absolute_P10_12 | 0.0161 |
| Relative_P26_28 | 0.0187 |
| KD_R1_Terrs | 0.0192 |
| Absolute_theta | 0.0199 |
| Relative_P16_18 | 0.0285 |
| Absolute_P24_26 | 0.0297 |
| Absolute_P56_58 | 0.0331 |
| Absolute_P22_24 | 0.0349 |
| Relative_P30_45 | 0.0363 |
| Absolute_alpha | 0.0392 |
| Absolute_P54_56 | 0.0451 |
| Skewness_F | 0.0474 |
| Relative_gamma | 0.0475 |

Table 2 illustrates significantly different features between concussed and control subjects during the Eyes Closed task as determined by the Wilcoxin test (in JMP Pro by SAS). Features only listed for those with False Positive Rate p-value p<0.05.

TABLE 2

| | Wilcoxin |
|---|---|
| Eyes Closed | FPR p-value |
| Extracted Feature | Prob > ChiSq |
| KD_R1_C1_Secs | 0.0001 |
| Relative_P18_20 | 0.0003 |
| Relative_P34_36 | 0.0004 |
| KD_R1_Tsecs | 0.0007 |
| Relative_P32_34 | 0.0010 |
| Relative_theta/Relative_beta | 0.0010 |
| Absolute_theta/Absolute_beta | 0.0010 |
| KD_R2_C1_Secs | 0.0013 |
| Relative_P20_22 | 0.0013 |
| Relative_P30_45 | 0.0013 |
| Relative_P12_20 | 0.0017 |
| KD_R1_C3_Secs | 0.0017 |
| Relative_gamma | 0.0020 |
| Kurtosis_F | 0.0021 |
| Relative beta | 0.0024 |
| KD_R1_C2_Secs | 0.0027 |
| Relative_P14_16 | 0.0036 |
| Relative_P38_40 | 0.0039 |
| Relative_P26_28 | 0.0045 |
| StdDev_F | 0.0053 |
| Skew_F | 0.0058 |
| Relative_P16_18 | 0.0064 |
| KD_R2_Tsecs | 0.0066 |
| KD_R2_C3_Secs | 0.0066 |
| Relative_P36_38 | 0.0107 |
| Relative_P40_42 | 0.0108 |
| KD_Fsecs | 0.0150 |
| Relative_P22_24 | 0.0160 |
| Relative_P28_30 | 0.0160 |
| Relative_P44_46 | 0.0177 |
| Relative_P20_30 | 0.0186 |
| KD_R2_C2_Secs | 0.0208 |
| Relative_P12_14 | 0.0222 |
| Relative_P42_44 | 0.0289 |
| Relative_delta/Relative_beta | 0.0292 |
| Absolute_delta/Absolute_beta | 0.0292 |
| Mean_F | 0.0305 |
| Relative_P30_32 | 0.0438 |

Table 3 illustrates significantly different features between concussed and control subjects during the Eyes Open task as determined by Analysis of Variation (ANOVA) or equivalently Student's t-test for two groups (in JMP Pro by SAS). Features only listed for those with False Positive Rate p-value p<0.05.

TABLE 3

| | ANOVA |
|---|---|
| Eyes Open | FPR p-value |
| Extracted Feature | Prob > F |
| KD_R1_C1_Secs | 0.00006 |
| Relative_P18_20 | 0.00014 |
| Relative_P20_22 | 0.00015 |
| Relative_P34_36 | 0.00016 |
| Relative_P32_34 | 0.00020 |
| Absolute_theta/Absolute_alpha+beta | 0.00020 |
| Relative_theta/Relative_alpha+beta | 0.00020 |
| KD_R1_Tsecs | 0.00030 |
| Relative_P30_45 | 0.00033 |
| Relative_P12_20 | 0.00036 |
| KD_R1_C3_Secs | 0.00037 |
| Relative beta | 0.00046 |
| Relative_P26_28 | 0.00049 |
| Relative_gamma | 0.00053 |
| StdDeviation_F | 0.00064 |
| Relative_theta/Relative_beta | 0.00066 |
| Absolute_theta/Absolute_beta | 0.00066 |
| KD_R1_C2_Secs | 0.00130 |
| Relative_P28_30 | 0.00132 |
| Relative_P38_40 | 0.00143 |
| KD_Final_secs | 0.00150 |
| Relative theta | 0.00153 |
| Relative_P16_18 | 0.00219 |
| Kurtowsis_F | 0.00220 |
| Relative_theta/Relative_alpha | 0.00228 |
| Absolute_theta/Absolute_alpha | 0.00228 |
| Relative_P14_16 | 0.00235 |
| Relative_P36_38 | 0.00245 |
| KD_R2_C1_Secs | 0.00287 |
| KD_R2_Tsecs | 0.00297 |
| Relative_P40_42 | 0.00309 |
| KD_R2_C3_Secs | 0.00353 |
| Relative_P6_8 | 0.00448 |
| Skewness_F | 0.00457 |
| Relative_P20_30 | 0.00614 |
| Mean_F | 0.00653 |
| Relative_P4_6 | 0.00736 |
| Relative_P30_32 | 0.00780 |
| Relative_P12_14 | 0.00787 |
| KD_R2_C2_Secs | 0.00809 |
| Relative_P44_46 | 0.00879 |
| Relative_delta/Relative_beta | 0.01569 |
| Absolute_delta/Absolute_beta | 0.01569 |
| Relative_P42_44 | 0.01816 |
| Relative_P24_26 | 0.01887 |
| KD_R1_Terrs | 0.01918 |
| Relative_theta+delta/Relative_alpha+beta | 0.02155 |
| Absolute_theta+delta/Absolute_alpha+beta | 0.02155 |
| Relative_P46_48 | 0.04040 |

Table 4 illustrates significantly different features between concussed and control subjects during the Eyes Open task as determined by the Wilcoxin test (in JMP Pro by SAS). Features only listed for those with False Positive Rate p-value p<0.05.

TABLE 4

| | Wilcoxin |
|---|---|
| Eyes Open | FPR p-value |
| Extracted Feature | Prob > ChiSq |
| Relative_P32_34 | 0.00005 |
| Relative_P34_36 | 0.00008 |
| Relative_theta/Relative_beta | 0.00010 |
| Absolute_theta/Absolute_beta | 0.00010 |
| KD_R1_C1_Secs | 0.00010 |
| Relative_P30_45 | 0.00015 |
| Relative_P20_22 | 0.00022 |
| Relative_gamma | 0.00027 |
| Relative_P18_20 | 0.00029 |
| Relative_P26_28 | 0.00041 |
| Relative_P38_40 | 0.00047 |
| Kurtosis_F | 0.00053 |
| Relative beta | 0.00071 |
| KD_R1_Tsecs | 0.00073 |
| Relative_theta/Relative_alpha+beta | 0.00074 |
| Absolute_theta/Absolute_alpha+beta | 0.00074 |
| Relative_P12_20 | 0.00081 |
| StdDev_F | 0.00103 |
| KD_R2_C1_Secs | 0.00131 |
| Relative_P28_30 | 0.00154 |
| KD_R1_C3_Secs | 0.00166 |
| Skewness_F | 0.00194 |
| Relative_P40_42 | 0.00202 |
| Relative_P36_38 | 0.00208 |
| Relative_P16_18 | 0.00230 |
| Relative_P20_30 | 0.00240 |
| KD_R1_C2_Secs | 0.00265 |
| Relative_P30_32 | 0.00283 |
| Relative_P22_24 | 0.00325 |
| Relative_delta/Relative_beta | 0.00461 |
| Absolute_delta/Absolute_beta | 0.00461 |
| Relative_thetata | 0.00576 |
| Relative_P24_26 | 0.00656 |
| KD_R2_Tsecs | 0.00658 |
| KD_R2_C3_Secs | 0.00658 |
| Relative_P4_6 | 0.00681 |
| Relative_theta+delta/Relative_alpha+beta | 0.00823 |
| Absolute_theta+delta/Absolute_alpha+beta | 0.00823 |
| Relative_P6_8 | 0.00866 |
| Relative_P14_16 | 0.00944 |
| Mean_F | 0.00967 |
| Relative_P44_46 | 0.01093 |
| KD_Fsecs | 0.01500 |
| Relative_P42_44 | 0.01635 |
| Relative_P12_14 | 0.02078 |
| KD_R2_C2_Secs | 0.02079 |
| Relative_theta/Relative_alpha | 0.02272 |
| Absolute_theta/Absolute_alpha | 0.02272 |
| Relative_delta/Relative_alpha+beta | 0.02428 |
| Absolute_delta/Absolute_alpha+beta | 0.02428 |

Stepwise logistic regression to build a predictive model to classify subjects into either the concussed or control groups (in JMP Pro by SAS) identified several extracted features useful for prediction from the Eyes Closed first task. The best model (that which minimized the Bayesian Information Criterion (BIC)—see Hastie et al., “Elements of Statistical Learning: Data Mining, Inference, and Prediction,” Springer, 2^{nd} Edition, 2009, Section 7.7, p. 233) included the {Kurtosis_F, Relative_P4_{—}6, Relative_P6_{—}8, Relative_P18_{—}20, Relative_P24_{—}26, Relative_P32_{—}34, Relative_P36_{—}38, KD_R1_C1_Secs, KD_R1_C2_Secs}. This logistic regression model achieved an Receiver Operator Characteristic curve Area Under the Curve (ROC AUC) of 0.9935 getting 24 concussed correct (TP=True Positive), 41 control correct (TN=True Negative), 1 concussed wrong (FN=False Negative), and 2 controls as concussed (FP=False Positive) for an overall accuracy (TP+TN)/(total_P+total_N) of (24+41)/68=95.6% accuracy. When reducing the number of features used in the model down to the five most important (using stepwise logistic regression), the model consisting of {Relative_P18_{—}20, Relative_P24_{—}26, Relative_P32_{—}34, Relative_P36_{—}38, and KD_R1_C1_Secs} produced an ROC AUC=0.9107 with 19 TP, 39 TN, 6 FN, 4 FP or an overall accuracy of (19+39)/68=85%. As one skilled in the art appreciates, the most important consideration is the reduction of the number of False Negatives (FN) which puts the brain at risk again for further injury.

Using an alternate modeling technique, stepwise Linear Discriminant analysis, the top five predictive factors from the Eyes Closed first task included {KD_R1_C1_Secs, Relative_P18_{—}20, Relative_P24_{—}26, Relative_P32_{—}34, Relative_P36_{—}38} which achieved an ROC AUC=0.8897 with 22 TP, 35 TN, 3 FN, 8 FP for an overall accuracy of (22+35)/68=84%.

Repeating the same analysis on the Eyes Open second task yields the following results. Using stepwise logistic regression to build a predictive model to classify subjects into either the concussed or control groups (in JMP Pro by SAS) identified several extracted features useful for prediction from the Eyes Open second task. The best model (that which minimized the BIC) included {Peak_F, Mean_F, Kurt_F, Relative_beta, Relative_P22_{—}24, Relative_P28_{—}30, Relative_P32_{—}34, Relative_theta/Relative_beta, KD_R1_C1_Secs}. This logistic regression model achieved a Receiver Operator Characteristic curve Area Under the Curve (ROC AUC) of 1.000 getting 25 concussed correct (TP=True Positive), 43 control correct (TN=True Negative), 0 concussed wrongly identified as controls (FN=False Negative), and 0 controls wrongly identified as concussed (FP=False Positive) for an overall accuracy of (25+43)/68=100%. When reducing the number of features used in the model down to the five most important (using stepwise logistic regression), the model consisting of {Peak_F, Relative_beta, Relative_P22_{—}24, Relative_theta/Relative_beta, KD_R1_C1_Secs}produced an ROC AUC=0.88186 with 17 TP, 38 TN, 8 FN, 8 FP for an accuracy of (17+38)/68=81%.

Using an alternate modeling technique, stepwise Linear Discriminant analysis, the top five predictive factors from the Eyes Open second task included {KD_R1_C1_Secs, Peak_F, Relative_P22_{—}24, Relative_P34_{—}36, Relative_theta/Relative_beta} which achieved an ROC AUC=0.8726 with 15 TP, 41 TN, 10 FN, 2 FP for an accuracy of (15+41)/68=82%.

For those skilled in the art, it is a direct calculation to compute the sensitivity (Sens)=TP/(TP+FN), specificity (Spec)=TN/(FP+TN), positive predictive value (PPV)=TP/(TP+FP) and negative predictive value (NPV)=TN/(TN+FN) from the truth table (sometimes called a confusion matrix). The predictive models reported above on the study subjects are exemplary of the predictive signatures and their clinical performance. Adjustment of the features used to include any of those listed in Table 1 through Table 4 are contemplated as other embodiments of the present invention. Moreover, it should be clear to one skilled in the art that models of a subset of these predictive features are also covered by the present invention.

As one adds additional modalities of information, from either the accelerometers, the microphone from voice analysis, from the camera or biosensor for image analysis, one can anticipate that the accuracy of such predictive models will increase further as it aids healthcare providers in the diagnosis of a given condition. Those skilled in the art will appreciate that tables of biometric extracted features such as those illustrated in Tables 1-4 above may be generated by processing devices to extract candidate features from the multiple received biological sensor data from which multi-modal predictive signatures can be created, verified, and ultimately validated which correlate with brain health, disease, and injury to thereby provide a multi-modal system to assess brain health and function.

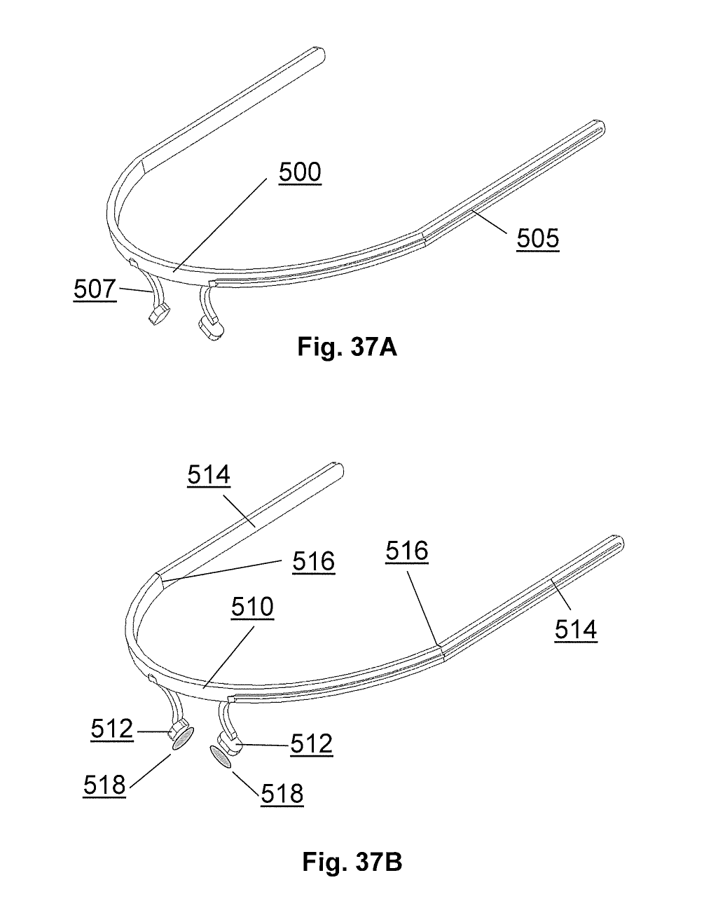

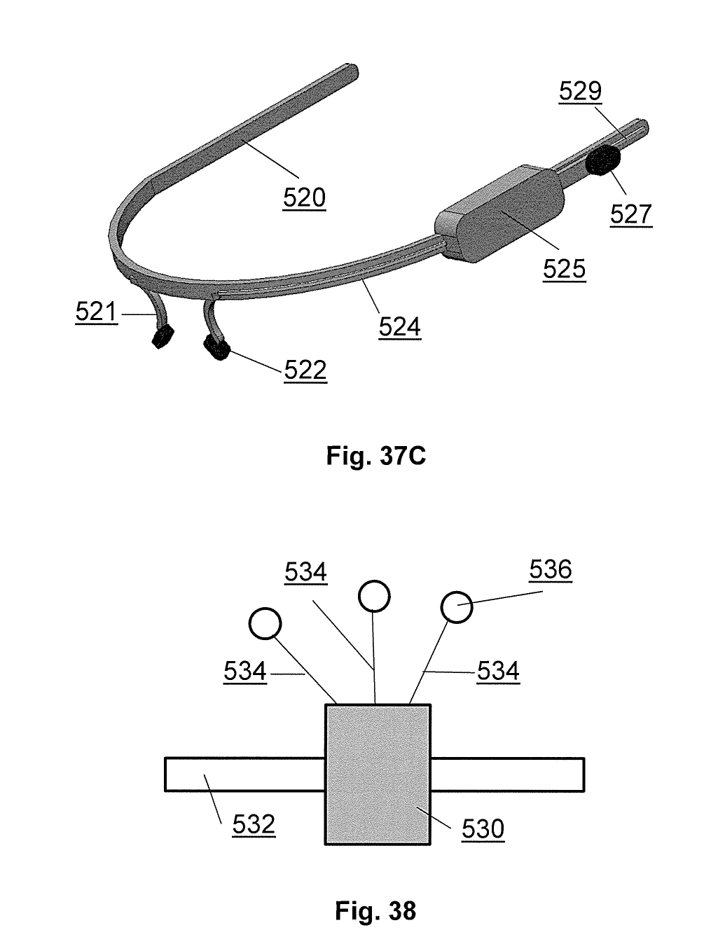

FIG. 37A illustrates an alternate REM support to be worn on the human skull in the form of an eyeglasses frame without the lens, a lens-less eye glass frame. The frame 500 can have temples 505 which rest on the ears and nose supports 507 which rest on the nose. In one embodiment of the present invention, a disposable single piece eye glass frame in the form of 500 can be utilized to support an REM that is supported in the front at position 500 or alternatively along the side at position 505. A keyed channel can be employed which creates a tailored fit to the REM to slide along the temple from rear to forward to sit away and off the face. Electrodes to the skull and electrodes to mastoid can be situated adjacent to the lens-less eye glass frame.

FIG. 37B is an alternate embodiment of the lens-less eye glass frame where in this case reusable frame 510 with nose supports 512 have disposable temples 514 which connect to the reusable frame 510 at connection point 516 as well as disposable nose pads 518. In a modification to the present embodiment, there can be wires either laminated to the external surfaces or molded within which connect to wires which run down nose supports 512 to make electrical contact with electrically conducting disposable nose pads 518 which can serve as mastoid REF reference and GND ground.

FIG. 37C is an alternate embodiment of the lens-less eye glass frame where in this case reusable frame 524 with nose supports 521 can have either disposable temples 520 or disposable sleeves that slip over the ends of the temples to provide a protective sheath between the device and the subject, as so called applied part. In this case, wire 529 can run down the outside of the frame 524 to one nose support with electrical contact to conducting disposable nose pad 522 while a second wire on the inside of the frame can run along the inside to the other nose support 521 and make electrical contact with the disposable nose pad on the end of nose support 521. REM 525 can slide along either temple to a position where it does not come in contact with the subject. If needed, electrode 527 can connect to mastoid at the ear or alternatively, electrode 527 can move forward to 10-20 electrode position Fp1. One skilled in the art will appreciate that running both nose-piece conductors on the same side of the frame 524 with the active electrode lead wire on the inside is fully contemplated as would be three adjacent wires along one surface of the lens-less eye glass frame. The same would be true for more than one active electrode as particularly needed in any given circumstance.

FIG. 38 is an alternate embodiment of REM 530 taking the shape of a rectangular unit which is attached to the body, as a non-limiting example around the upper arm or around the waist, with strap 532. At the output of the REM device, are 3 disposable leads 534 with electrodes 536 attached at the end of each. Two of these can serve as reference REF and ground GND. Equally, a fourth, fifth and additional leads are equally contemplated as equivalent to the non-limiting embodiment shown.

### EXAMPLES

While the above description contains many specifics, these specifics should not be construed as limitations on the scope of the invention, but merely as exemplifications of the disclosed embodiments. Those skilled in the art will envision many other possible variations that are within the scope of the invention. The following examples will be helpful to enable one skilled in the art to make, use, and practice the invention.

### Example 1

### Creation of a Remote Calibration Cable Assembly for Remote Quality Control Purposes

Using a soldering iron, resistors, stereo jack pin, wire and alligator clips, a calibration and quality control cable was constructed. The voltage divider included an upper ¼ watt resistor of 100 ohms (Ω) and a lower ¼ watt resistor of 1,000,000 ohms or 1 MΩ to divide the reference signals down by a factor of 10⁴ from 1 volt to 100 μv and 50 mV to 5 μV. These stepped down signals are thus within the typical physiological range of a 1 μV to 100 μV and thus useful for assessment and calibration of EEG systems. If desired, metal film resistors with tighter tolerances could be used. This cable may be attached to an REM output and directly wired to contact the REM inputs to calibrate and confirm the system is working. Alternatively, this same design may be engineered to reside on an internal printed circuit board and to confirm system calibration internally without use of an external cable. This approach simplifies the procedure but does not test for the integrity of the leads going into the REM and thus confirms the electronics but not as much as including the leads of the system.

### Example 2

### Use of Independent Accelerometers to Confirm Balance and Posture

A pair of USB Accelerometer Model X6-1A electronic REM modules were purchased from Gulf Coast Data Concepts. Experiments were conducted with these 3-axis accelerometers and used while conducting assessments of human motion and stability. The data acquisition and display software was installed on a Dell Latitude E6520 laptop. Analysis was conducted in JMP Pro v10 from SAS. Features of these accelerometers include the fact that they transfer data through any USB port of a laptop, they have a user selectable +/−2 g acceleration range, and they have a user selectable sample rate of 10, 20, 40, 80 or 160 Hertz, with either 12 bit or 16 bit resolution internal to the REM. Experiments were carried out while the REM was attached with an elastic wrist band or ankle band for simultaneous two location human motion data capture and stability analysis.

In an effort to field calibrate the accelerometers before each use, the inventors devised the means of suspending the accelerometer from a fixed length of string such that it is well known that the period of a simple pendulum is two times pi times the square root of the length L of the pendulum divided by the constant of gravity g. If the same cord were used over and over, this would permit a relative calibration to be sure measurements were internally precise from experiment to experiment. In FIG. 33, one can see three traces collected in a single 3-axis MEMS accelerometer used as a pendulum to calibrate the device. Trace 380 shows a decaying sinusoidal oscillation in the x-axis from the fixed length pendulum while traces 385 for the y-axis and 390 for the z-axis show little to no oscillation. Alternatively, a second method was employed using a fixed frequency oscillator in the form of a mechanical massage device that oscillates at fixed frequency. This too worked as a means to calibrate the accelerometer although it required keeping a battery available and transporting the device. Either the simple pendulum or the electronically controlled mechanical oscillator may be used to calibrate an REM embedded accelerometer or a peripheral MCU accelerometer.

The accelerometer could also include a 3 axis or less gyrometer and 3 axis or less digital compass as well as other biosensors or motion processors that are combined into an integrated circuit to be incorporated into single chip or multiple chip arrangements. A non-limiting example would be an Invensense MPU-9150 Nine-Axis (Gyro+Accelerometer+Compass) MEMS MotionTracking™ Device. Moreover, as the accelerometer or multi-axis motion processing unit (MPU) can be embedded into the REM, this configuration would include the MPU in the head based REM. An alternate configuration would include the accelerometer or MPU in a peripheral REM that could be wrist based, ankle based, small of the back based, or other key body location away from the head based REM.

Once the device was calibrated, the inventors experimented with the motion based measurements by attaching one REM accelerometer to a wrist and a second one to the contralateral ankle of a human subject with one inch wide elastic bands. Various obstacles were placed in the path around a central conference table to insure that the human subject would have to avoid obstacles and register accelerations. In FIG. 34, one can see data collected from a human subject who was wearing the 3-axis accelerometer on his wrist as he swung his arm back and forth while walking through an obstacle course in his laboratory. Trace 400 shows the x-axis, 405 shows the y-axis and trace 410 shows the z-axis. Time runs along the x-axis and the acceleration in each direction is plotted along the y-axis of each trace. To the far right, summary statistical analysis of the time series is presented in addition to the ability to look for individual features.

### Example 3

### TIRHR Concussion Study

In collaboration with an non-profit mountain based medical institute near Lake Tahoe, two groups of subjects were enrolled in an Institutional Review Board approved clinical protocol, wherein the first group of subjects (group A) were clinically diagnosed with a concussion (mTBI) or mild traumatic brain injury and second control cohort of subjects (group B) were enrolled who did not have any issue with concussion and served as Controls (CTL) were recruited under the supervision of an Institutional Review Board. Participants from both groups A and B were scanned identically with an electronic REM module including a single electrode EEG device as described in PCT Patent Application PCT/US2012/046723, filed Jul. 13, 2012. The 5 minute scan protocol included 30 seconds Eyes Closed, 30 seconds Eyes Open, approximately 3 minutes to conduct the King-Devick test and then closed with a 30 seconds Eyes Closed, 30 seconds Eyes Open block again. The stop watch times and errors for each card of the King-Devick test were recorded manually by the test administrators while the peripheral MCU (a laptop computer) presented the cards and recorded the responses of the individuals via the microphone. The head based REM module continuously recorded the forehead EEG from position Fp1 relative to mastoid on the ear for reference REF and ground GND. The data was encrypted locally before being transported over a secure pipe to a virtual server in cyberspace.

Signal analysis scientists were blinded to participant clinical diagnosis for the purposes of artifact detection, signal processing and feature extraction. The extracted feature data table was then quality controlled and scrubbed to remove as many errors as possible. The total time for the King-Devick test was calculated according to the published procedure of using the minimal number of errors and then summing the individual times to read all three cards in succession. This total time represents one extracted variable and underwent a logistic classification model. The result of this model indicated that the King-Devick total time in seconds alone predicted the classification of the individuals approximately 62% of the time (AUC=0.62).

Independently, analysis for the parallel data stream of EEG brainwave information, sampled at 128 samples per second with 10-bits of amplitude resolution was then Fourier transformed to determine the spectral properties. The relative power in each of the delta, theta, alpha, beta and gamma bands was analyzed in a logistic classification model where the EEG feature was the predictor x-variable and the clinical outcome (grp A or B) was the outcome y-variable in the model. The analysis was conducted in JMP Pro v10 from SAS (Cary, N.C.).

In FIG. 35, one can see the logistic plot 420 for the relative-beta power (from 12-30 Hz) showing a decreased relative beta power in the concussed group A relative to control group B. When one constructs the receiver operating characteristic (ROC) curve 430, one can see that the EEG feature alone predicts with accuracy approximately 65% of the time as defined by the summary AUC statistic. In FIG. 36, one can see in ROC plot 440 that the area under the curve (AUC) is now 70% when the King-Devick test time (a cognitive measure of the subjects brain) is combined with the relative beta EEG power (a brainwave measure), creating a multi-modal signature. When one adds the co-variates of age and gender, the AUC raises to 76% as shown in ROC plot 450, fully corroborating the system and methods of the invention. As one adds additional modalities of information, from either the accelerometers, the microphone from voice analysis, from the camera or biosensor for image analysis, one can anticipate that the accuracy of the predictive model will increase further as it aids healthcare providers in the diagnosis of a given condition. This exemplifies the power of a multi-modal system to assess brain health and function.

Importantly, Tables 1, 2, 3, and 4 above identify extracted features for use in predictive models to classify new subjects into either the concussed or control groups. Tables 5 and 6 show the results of one such model building using stepwise logistic regression. It should be clear to one skilled in the art that models constructed from a subset of these predictive features are also covered by the present invention.

### Example 4

### Lehigh Concussion Study

In collaboration with an NCAA Division 1 university, several groups of subjects were enrolled in an Institutional Review Board approved clinical protocol, wherein the first group of subjects (group A) were clinically diagnosed with a concussion (mTBI) or mild traumatic brain injury, a second control cohort of subjects (group B) were enrolled who did not have any issue with concussion and served as Controls (CTL), and other athletes from other sports (Group C, etc.) were recruited under the supervision of an Institutional Review Board as well. Participants from groups A, B, C and others were scanned identically with an electronic REM module including a single electrode EEG device as described in US Patent Application PCT Patent Application PCT/US2012/046723, filed Jul. 13, 2012. The 22-24 minute scan protocol included 1 minute of Eyes Closed, 1 minute of Eyes Open, an automated application of the Graded Symptom Checklist, elements of the Standard Assessment of Concussion (SAC) including memory, concentration, delay recall, a full Balance Error Scoring System (on both firm and foam surfaces), King-Devick Test Cards, binaural beat audio stimulation at 6 and 12 hertz beat frequency centered at 400 Hz, photic stimulation, and a fixation task including a moving red cross for 1 minute. The stop watch times and errors for each card of the King-Devick test were recorded manually by the test administrators while the peripheral MCU (a laptop computer) presented the cards and recorded the responses of the individuals via the microphone. The BESS errors were recorded manually as well as the SAC responses. The head based REM module continuously recorded the forehead EEG from position Fp1 relative to mastoid on the ear for reference REF and ground GND. An EEG data stream, a cognitive data stream (reaction time and accuracy), and a microphone data stream were recorded depending upon which tasks were being conducted. The data was encrypted locally before being transported over a secure pipe to a virtual server in cyberspace.

Signal analysis scientists were blinded to participant clinical diagnosis for the purposes of artifact detection, signal processing and feature extraction. The extracted feature data table was then quality controlled and scrubbed to remove as many errors as possible. The total time for the King-Devick test was calculated according to the published procedure of using the minimal number of errors and then summing the individual times to read all three cards in succession. This total time represents one extracted variable and underwent a logistic classification model. Serial assessments were conducted on both concussed athletes and controls with up to eight or nine scans assessing both concussed and controls.

### Example 5

### Rothman Concussion Study

In collaboration with a clinical practice and a concussion expert, two groups of subjects were enrolled in an Institutional Review Board approved clinical protocol, wherein the first group of subjects (group A) were clinically diagnosed with a concussion (mTBI) or mild traumatic brain injury and a second control cohort of subjects (group B) were enrolled who did not have any issue with concussion and served as Controls (CTL) and were recruited under the supervision of an Institutional Review Board. Participants from both groups A and B were scanned identically with an electronic REM module including a single electrode EEG device as described in PCT Patent Application PCT/US2012/046723, filed Jul. 13, 2012. The 25 minute scan protocol included 1 minute Eyes Closed, 1 minute Eyes Open, and then approximately 25 minutes of scanning while the student athlete completed the ImPACT computer test with a head electronic REM module streaming EEG data to a nearby peripheral MCU (Dell Vostro 3550 laptop). Key clicks on the peripheral MCU laptop indicated the temporal beginning and ending of each of the various tasks within the ImPACT computer assessment. This represents another multi-modal assessment combining neuropsychological testing, EEG, and clinical observation in accordance with the invention.

Those skilled in the art will also appreciate that the invention may be applied to other applications and may be modified without departing from the scope of the invention. For example, the signal processing described herein may be performed on a server, in the cloud, in the electronics module, or on a local PC, tablet PC, smartphone, or custom hand held device Accordingly, the scope of the invention is not intended to be limited to the exemplary embodiments described above, but only by the appended claims.

What is claimed:

1. A system for capturing multiple streams of biological sensor data for assessing brain health of a user, comprising:
a plurality of biological sensors adapted to collect biological sensor data from the user, said biological sensors including an active brainwave sensor that collects at least one channel of EEG brainwave data, and at least one of the following:
an accelerometer and/or a gyrometer that collects motion, position, and stability data to provide quantitative stability and balance measurements,
a peripheral sensing device that collects cognitive information in the form of neuropsychological data comprising key board keystrokes, mouse clicks, and/or touch panel events to convey reaction time and accuracy information,
a microphone that records human speech to capture verbal responses of the human subject during a battery of tasks to either cognitive challenges or auditory stimulations, and
a camera or biosensor that records that records eye movements, eye saccade and other biometric identification information;
an electronic module that simultaneously records biological sensor data collected by said plurality of biological sensors; and
a stimulation device that applies at least one of a visual stimulant, an auditory stimulant, a gastronomic stimulant, an olfactory stimulant, and/or a motion stimulant to the user, wherein the plurality of biological sensors simultaneously measure the body's response to stimulants applied by said stimulation device for recordation by said electronic module.

2. A system as in 1, further comprising means for transmitting the biological sensor data collected by said electronic module to a remote processing device.

3. A system as in claim 2, wherein said remote processing device processes biological sensor data received from said electronic module to identify and characterize artifacts, to extract candidate features for classification and storage and/or for comparison to previously acquired candidate features, and to generate a report.

4. A system as in claim 3, wherein the remote processing device further builds extracted biometric tables from candidate features extracted from received biological sensor data.

5. A system as in claim 4, wherein the remote processing device is further programmed to construct predictive signatures including candidate features extracted from multiple biological sensor data streams, said predictive signatures correlating with multi-modal signatures of brain health, disease and injury.

6. A system as in claim 1, wherein said peripheral sensing device, microphone, and camera or biosensor are implemented in a PC, tablet PC, smartphone or custom hand held device.

7. A system as in claim 6, wherein said PC, tablet PC, smartphone or custom hand held device is programmed by software that causes said PC, tablet PC, smartphone or custom hand held device to administer instructions to the user via a sound card and/or visual display of the PC, tablet PC, smartphone or custom hand held device.

8. A system as in claim 6, wherein said PC, tablet PC, smartphone or custom hand held device is further programmed by software that provides control signals to said stimulation device.

9. A system as in claim 1, wherein said plurality of biological sensors further include a heart rate sensor that monitors heart rate, a pulse oximeter that measures arterial oxygenation, a temperature sensor that measures body temperature, a galvanic skin response or electrodermal response sensor that measures skin surface galvanic skin conductance and/or electrical skin resistance, means for assessing cerebral blood perfusion, and/or means for assessing vaso-motor reactivity.

10. A system as in claim 9, wherein at least one of said heart rate sensor, said pulse oximeter, said temperature sensor, and said galvanic skin response or electrodermal response sensor is incorporated into a peripheral electronic module separate from said electronic module.

11. A system as in claim 1, further comprising a disposable headband adapted to mount said electronic module.

12. A system as in claim 1, further comprising a glasses frame adapted to mount said electronic module.

13. A system as in claim 12, wherein said glasses frame has disposable ear temple supports and disposable nose pads.

14. A system as in claim 12, wherein said glasses frame includes integrated wires adapted to connect to at least one biological sensor.

15. A method for capturing multiple streams of biological sensor data for assessing brain health of a user, comprising:
using a stimulation device to apply at least one of a visual stimulant, an auditory stimulant, a gastronomic stimulant, an olfactory stimulant, and/or a motion stimulant to the user;
a plurality of biological sensors simultaneously measuring the body's response to stimulants applied by said stimulation device, said plurality of biological sensors adapted to collect at least one channel of EEG brainwave data, and at least one of the following:
motion, position, and stability data to provide quantitative stability and balance measurements,
cognitive information in the form of neuropsychological data comprising key board keystrokes, mouse clicks, and/or touch panel events to convey reaction time and accuracy information,
human speech for capturing verbal responses of the human subject during a battery of tasks to either cognitive challenges or auditory stimulations, and
eye movements, eye saccade and other biometric identification information; and
recording biological sensor data collected by said plurality of biological sensors in an electronic module.

16. A method as in 15, further comprising transmitting the biological sensor data collected by said electronic module to a remote processing device.

17. A method as in claim 16, further comprising processing received biological sensor data to identify and characterize artifacts, to extract candidate features for classification and storage and/or for comparison to previously acquired candidate features, and to generate a report.

18. A method as in claim 17, further comprising building extracted biometric tables from candidate features extracted from received biological sensor data.

19. A method as in claim 18, further comprising constructing predictive signatures including candidate features extracted from multiple biological sensor data streams, said predictive signatures correlating with multi-modal signatures of brain health, disease and injury.

20. A method as in claim 15, further comprising a PC, tablet PC, smartphone or custom hand held device administering instructions to the user via a sound card and/or visual display of the PC, tablet PC, smartphone or custom hand held device.

21. A method as in claim 20, further comprising said PC, tablet PC, smartphone or custom hand held device providing control signals to said stimulation device.

22. A method as in claim 15, further comprising collecting heart rate data, arterial oxygenation data, body temperature data, cerebral blood perfusion data, vaso-motor reactivity data, and/or skin surface galvanic skin conductance and/or electrical skin resistance data at said electronic module for recording with said biological sensor data.
